(19) United States
(12) Patent Application Publication
(10) Pub. No.: US 2025/0366890 A1
(43) Pub. Date: Dec. 4, 2025

(54) Stemless Semi-Constrained Implantable Joint Replacement Device

(71) Applicant: Djit Medtech, Inc., Baltimore, MD (US)

(72) Inventors: Marcia Andrews Hart, Baltimore, MD (US); Ryan David Katz, Millersville, MD (US); Fraser M. Smith, Salt Lake City, UT (US)

(21) Appl. No.: 19/304,437

(22) Filed: Aug. 19, 2025

Related U.S. Application Data
(63) Continuation of application No. 18/826,141, filed on Sep. 5, 2024.
Application No. 18/826,141 is a continuation-in-part of application No. PCT/US2023/016369, filed on Mar. 27, 2023.
(60) Provisional application No. 63/326,479, filed on Apr. 1, 2022.
(60) Provisional application No. 63/352,314, filed on Jun. 15, 2022.
(60) Provisional application No. 63/587,139, filed on Oct. 1, 2023.

(51) Int. Cl. A61B 17/70; A61B 17/84; A61F 2/46

(52) U.S. Cl. CPC A61B 17/7019; A61B 17/84; A61F 2/46

(57) ABSTRACT

A stemless implantable device for arthroplasty. In one example, the device can include a flexible connector having a proximal rod sleeve integrated in a proximal portion of the flexible connector and a distal rod sleeve integrated in a distal portion of the flexible connector. A proximal fixation rod can be configured to be inserted into the proximal rod sleeve. A distal fixation rod can be configured to be inserted into the distal rod sleeve. In another example, the device can include a proximal fixation hollow cylinder comprising a proximal opening and proximal flanges framing the proximal opening, and a distal fixation hollow cylinder comprising a distal opening and distal flanges framing the distal opening. A flexible connector can be configured to be held by the proximal flanges and the distal flanges.

### RELATED APPLICATIONS

This is a continuation application of U.S. application Ser. No. 18/826,141, filed Sep. 5, 2024, entitled “Stemless Semi-Constrained Implantable Joint Replacement Device”, which claims the benefit of U.S. Provisional Application Ser. No. 63/587,139, filed Oct. 1, 2023, and entitled, “Flex Stemless Semi-Constrained Implantable Joint Replacement Device,” which is also incorporated by reference in its entirety herein. This application further claims priority to PCT Application Serial No. PCT/US23/16369, filed Oct. 5, 2023, and entitled, “Stemless Semi-Constrained Implantable Joint Replacement Device,” which application claims the benefit of U.S. Provisional Application Ser. No. 63/326,479 filed on Apr. 1, 2022, and U.S. Provisional Application Ser. No. 63/352,314, filed Jun. 15, 2022, each of which are also incorporated by reference in their entirety herein.

### BACKGROUND

### Background

Arthritis of the interphalangeal joints due to osteoarthritis, rheumatoid arthritis, and traumatic injury affects nearly 20 million Americans over the age of 45. For example, in osteoarthritis, the degeneration of the joint can lead to bone-on-bone contact, which is a common cause of severe pain in the advanced stages of all forms of arthritis. Bone-on-bone contact leads to inefficient joint mechanics that impairs digital range of motion, accelerates the degenerative process, and may ultimately lead to an ankylosis or complete loss of motion at the joint. However, due to technical complexity and perceived market size, interphalangeal joint arthroplasty lags far behind arthroplasty of hips, knees, and shoulders. The present invention offers a solution to meet a conservatively estimated domestic market need of more than 1.2 million hand digit arthroplasty procedures per year with demand for approximately 1.4 joints replacements per procedure. Interphalangeal joints of the foot and other small joints of the human body and veterinary patients represent a secondary market that may be pursued. This technology opens the possibility of use in large joint arthroplasty, and non-human robotic and prosthetic joint systems as well.

Currently available solutions for chronic pain and stiffness in interphalangeal joints of the hand include arthroplasty, also known as joint replacement surgery, or fusion of the joint.

Fusion is a surgical treatment in which a portion of the opposing cartilaginous surfaces of adjacent phalangeal bones in the finger are eliminated and the prepared bones are then affixed to one in a prespecified position such that the bones will fuse together into a single osseous unit that is stable and pain-free. Due to the lack of durable and reliable arthroplasty alternatives, fusion remains the prevailing treatment for chronic pain in interphalangeal joints of the hand and results in permanent functional loss of movement at that joint.

In lieu of fusion one arthroplasty solution is a simple silicone hinge joint replacement device that was developed in the 1960's and is still in common usage today. This solution comprises a one-piece axial hinge formed of silicone in the shape of a flexible central node with opposed longitudinal stems on the proximal and distal faces of the central node. This configuration constrains flexion and extension along the sagittal axis of the finger. In some cases, a metal reinforcement plate (grommet) is integrated at the junction of the hinge node and stem for additional support at this high stress area within the device.

Insertion of the silicone hinge joint replacement device can be through a dorsal, lateral, or volar approach though typically involves a longitudinal incision on the dorsal aspect of the finger. This common dorsal approach necessitates surgical disruption of the extensor mechanism to allow for bone preparation. Preparation of the bone to receive the implant stems includes removal of a portion of the condyle head, and serially broaching the medullary canals of the proximal and middle phalanx to provide room for the implant stem. Because silicone hinge joint replacement implants are one piece and not modular this procedure is a suitable option for patients who are ligamentously deficient.

Another arthroplasty approach uses one of several types of unconstrained surface replacement devices with an individual proximal component and a separate distal component. Typical materials used for unconstrained multi-component arthroplasty include pyrocarbon and biocompatible ceramics. The proximal component head approximately replicates the intercondylar groove formation of the bicondylar joint and the distal component approximately replicates the interfacing intercondylar ridge that loosely glides within the opposing condylar groove. Unconstrained surface replacement options are suitable for patients who have strong ligamentous support to preserve the connection between the condylar groove and ridge. Good bone density and adequate girth as well as high quality soft tissues are required for secure implantation.

Alternatively, another approach for unconstrained surface replacement arthroplasty may employ a stemless system comprising a proximal head and a distal base that are held in place using shallow pins, natural osseointegration of biocompatible materials and compression maintained by the patient's ligaments and tendon structures. Stemless surface replacement options are suitable for patients with good bone stock and strong ligamentous support.

Modular small joint arthroplasty devices can be employed to reconstruct half of a joint in patients who have loss or damage to one joint surface with preservation of the other. This technique is referred to as a hemi-arthroplasty and can be used to resurface either the head of the proximal phalanx or the base of the middle phalanx.

There are a number of significant problems and disadvantages associated with the conventional devices described above.

The constrained silicone hinge joint replacement device is not designed to approximate physiologic motion and thus consistently fails in its ability to deliver predictable motion outcomes. Other disadvantages of this stemmed one-piece device include implant loosening, implant dislocation, implant breakage/silicone fragmentation, osteolysis and erosion through bone, and collagen encapsulation of the implant that can further restrict range of motion. Revision of the device to address failure or dissatisfaction is challenging due to osteolytic changes caused by unnatural forces imparted by the implant. Moreover, the complications associated with a failed revision may lead the patient down the path of amputation.

Because the unconstrained surface replacement arthroplasty (SRAs) implant has two separate components, joint stability is reliant upon implant shape and surrounding soft tissues, which makes this solution susceptible to dislocation and instability. Squeaking or other sounds due to direct contact between the components is a common complaint. Implant loosening, osteolysis and erosion through bone along the stem axis are also common modes of failure for these devices. Moreover, stemmed multi-component surface replacement are not recommended for patients with ligament deficiency, extensor tendon injuries, or poor bone stock. As with the constrained silicone implants, revision in the setting of failure or dissatisfaction is challenging often due to bone loss or weakening of the bone, and the complications associated with a failed revision may eventually lead to amputation.

Successful results with stemless multi-component options largely depend on healthy soft tissue structures and are therefore contraindicated for patients with ligamentous deficiency or extensor tendon injuries. However, because the stemless device implantation requires little bone preparation and therefore spares phalangeal bone from excision and unnatural stresses, revision options are more forgiving. Typically, sufficient bone remains after extraction of a failed or failing device to allow revision of remaining bone to receive a replacement, or to perform a successful fusion procedure, in lieu of amputation.

With all currently available interphalangeal arthroplasty devices, stiffness and unpredictable motion outcomes remains a major drawback. This may be due to a dearth of surrounding soft tissues, the proximity of the surgical incision to the joint, or post-surgical scarring that often restricts joint mobility through the buildup of fibrous tissue and interference with tendon function. In addition, extended immobilization often employed by surgeons to allow for postoperative soft tissue healing can result in stiffness, tendon adhesions and/or extension contractures thus limiting the potential of the patient to regain meaningful motion.

As a result of these issues, current implantable devices and surgical procedures often fail to predictably restore the range of motion expected by patients or desired by surgeons. Failure rates of current devices on the market remain unacceptably high. Thus, fusion has long been seen as the primary solution for pain relief and finger stability despite its serious and permanent functional consequences.

Given the absence of reliable and effective alternative implant options, disability and stiffness of injured or diseased finger joints is currently expected and tolerated.

### BRIEF SUMMARY OF VARIOUS EMBODIMENTS

The presently disclosed invention addresses the aforementioned problems and disadvantages of currently available treatments by providing a stemless semi-constrained implantable interphalangeal joint replacement device and a surgical method for implanting the same. The disclosure provides a stemless implantable device for arthroplasty, comprising: a flexible connector; optionally comprising a proximal transverse fixation system; and optionally comprising a distal transverse fixation system, optionally wherein the flexible connector is attached at one end to the proximal fixation system and attached at the other end to the distal fixation system. The disclosure provides a stemless implantable device for arthroplasty wherein said proximal transverse fixation system comprises a smooth or barbed rod. The disclosure provides a stemless implantable device for arthroplasty wherein said distal transverse fixation system comprises a smooth or barbed rod. The disclosure provides a stemless implantable device for arthroplasty wherein said proximal transverse fixation system and/or said distal transverse fixation system are identical. The disclosure provides a stemless implantable device for arthroplasty wherein said proximal transverse fixation system and/or said distal transverse fixation system are different. The disclosure provides a stemless implantable device for arthroplasty wherein said proximal fixation system and/or said distal fixation system independently or concurrently comprises a solid or hollow cylinder manufactured of a biocompatible material. The disclosure provides a stemless implantable device for arthroplasty wherein said proximal fixation system and/or said distal fixation system independently or concurrently comprises biocompatible materials selected from the group consisting of medical grade plastic, bioactive materials, carbon nanofibers, carbon plates, carbon strips, ceramic, composite materials, hydroxyapatite (HA) coatings, nickel-free super-elastic metal alloys, polyetheretherketone (PEEK), silicone, stainless steel, titanium alloys, titanium, ultra-high molecular weight polyethylene (UHMWPE), ceramic materials, nickel-based superalloys, cobalt-chromium alloys, nitinol alloys, other materials which may promote bone regeneration or materials derived therefrom, and combinations thereof. The disclosure provides a stemless implantable device for arthroplasty wherein said proximal fixation system and/or said distal fixation system independently or concurrently comprises a material which promotes bone regeneration. The disclosure provides a stemless implantable device for arthroplasty wherein said proximal fixation system and/or said distal fixation system independently or concurrently comprises material which promotes bone regeneration selected from the group consisting of medical grade plastic, bioactive materials, carbon nanofibers, carbon plates, carbon strips, ceramic, composite materials, hydroxyapatite (HA) coatings, nickel-free super-elastic metal alloys, polyetheretherketone (PEEK), silicone, stainless steel, titanium alloys, titanium, ultra-high molecular weight polyethylene (UHMWPE), other materials which may promote bone regeneration or materials derived therefrom, and combinations thereof.

The disclosure provides a stemless implantable device for arthroplasty wherein said proximal fixation system and/or said distal fixation system independently or concurrently are affixed within the bone using any fixation method. The disclosure provides a stemless implantable device for arthroplasty wherein said proximal fixation system and/or said distal fixation system independently or concurrently are affixed within the bone directly, with no rod or sleeve. The disclosure provides a stemless implantable device for arthroplasty wherein said proximal fixation system and/or said distal fixation system independently or concurrently are affixed within bone using a fixation method selected from the group consisting of cemented, uncemented, osseointegrated, osseointegrated with surface treatment or patterning to enhance bone ingrowth, press fit, threaded, fluted, capped, screw capped, pinned, other locking systems, and combinations thereof. The disclosure provides a stemless implantable device for arthroplasty wherein said proximal fixation system and/or said distal fixation system independently or concurrently comprises surface patterning to enhance natural bone ingrowth to anchor into bone. The disclosure provides a stemless implantable device for arthroplasty wherein said proximal fixation system and/or said distal fixation system independently or concurrently comprises a plurality of individual barbs of sufficient quantity and placement to confer stability within a patient's cancellous bone. The disclosure provides a stemless implantable device for arthroplasty wherein said proximal fixation system and/or said distal fixation system independently or concurrently comprises barbs configured and manufactured to break away under specific controlled mechanical action. The disclosure provides a stemless implantable device for arthroplasty wherein said proximal fixation system and/or said distal fixation system independently or concurrently comprises concentric fluting. The disclosure provides a stemless implantable device for arthroplasty wherein said proximal fixation system and/or said distal fixation system independently or concurrently comprises concentric fluting further wherein the fluting is pitched in a direction of insertion to prevent dislocation in a direction of extraction.

The disclosure provides a stemless implantable device for arthroplasty wherein said proximal fixation system and/or said distal fixation system independently or concurrently comprises a cannulated anchor comprising a hollow shaft and a slot, the slot being of sufficient width to allow both a first barrel shape at the proximal edge of the flexible connector and a second barrel shape at the distal edge of the flexible connector to pass through the slot and into a joint cavity; whereby said flexible connector will be held fast within the hollow shaft that incorporates a first thickened barrel shape at a proximal edge of said flexible connector; and a second thickened barrel shape at a distal edge of said flexible connector. The disclosure provides a stemless implantable device for arthroplasty wherein said proximal fixation system and/or said distal fixation system independently or concurrently comprises a cannulated anchor comprising a hollow shaft and a slot, the slot being of sufficient width to allow both a first barrel shape at the proximal edge of the flexible connector and a second barrel shape at the distal edge of the flexible connector to pass through the slot and into the joint cavity; whereby said flexible connector will be held fast within the hollow shaft that incorporates a first thickened barrel shape at a proximal edge of said flexible connector; a second thickened barrel shape at a distal edge of said flexible connector, wherein said proximal fixation system and/or said distal fixation system independently or concurrently comprises crimping flanges, wide flange screw, magnetic material, biometric monitoring capabilities, and/or bone scaffold integration.

The disclosure provides a stemless implantable device for arthroplasty wherein said proximal fixation system and/or said distal fixation system independently or concurrently comprises a rod which is affixed to a flexible connector with a cannulated screw anchor with longitudinal slot with an arc opening of approximately 0.2 radians. The disclosure provides a stemless implantable device for arthroplasty wherein said proximal fixation system and/or said distal fixation system independently or concurrently comprises a rod that is affixed to a flexible connector comprising a hollow cylinder with an arc opening of approximately 0.2 radians and with flanges substantially parallel to one another protruding from the cylinder edges, the flanges configured to secure a flexible connector within the sectional profile of the hollow cylinder. The disclosure provides a stemless implantable device for arthroplasty wherein said proximal fixation system and/or said distal fixation system independently or concurrently comprises a maximum length which does not exceed the intracortical dimension measured in the coronal plane of a patient's bone at the site of implantation. The disclosure provides a stemless implantable device for arthroplasty wherein said proximal fixation system and/or said distal fixation system independently or concurrently comprises a maximum dimension measured in the sagittal plane not to exceed approximately 70% of bone dimension measured in the sagittal plane at the site of implantation into a patient after sectioning or debridement, so as to allow sufficient residual bone to prevent bone fracture under normal force loading. The disclosure provides a stemless implantable device for arthroplasty wherein said proximal fixation system and/or said distal fixation system independently or concurrently comprises one or more crimping flanges for affixing a proximal fixation rod and/or a distal fixation rod to said flexible connector. The disclosure provides a stemless implantable device for arthroplasty wherein said proximal fixation system and/or said distal fixation system independently or concurrently comprise osseointegrated surface treatment. The disclosure provides a stemless implantable device for arthroplasty wherein said flexible connector is attached directly into prepared bone. The disclosure provides a stemless implantable device for arthroplasty wherein said flexible connector is attached into bone using a rod and sleeve, is attached into bone using an anchor, and/or is attached into bone using a cannulated anchor.

The disclosure provides a stemless implantable device for arthroplasty wherein said proximal fixation system and/or said distal fixation system comprises an anchor device configured in any shape selected from the group consisting of smooth, barbed, break-away barb, threaded screw, threaded screw with various pitch, threaded screw with various head type, threaded screw with various shank diameter, threaded screw with various thread diameters, threaded screw with various tip and crest profile, threaded screw with various thread angle, concave fluting, concentric fluting, pitched fluting, longitudinal fluting, a cap mechanically fastened to cortical bone using screws or pins, a screw cap, a hinged cap, a cuff-link cap, a magnetic cap, an osseointegrated bone cap, and combinations thereof, wherein said anchor device is configured to engage cortical bone at the implantation face only, or additionally engages cortical bone at the opposite end of said anchor device. The disclosure provides a stemless implantable device for arthroplasty wherein said flexible connector is attached to bone using an anchor. The disclosure provides a stemless implantable device for arthroplasty wherein said flexible connector is attached to bone using a cannulated anchor. The disclosure provides a stemless implantable device for arthroplasty wherein said flexible connector is attached to bone using a cannulated anchor, wherein the cannulated anchor further comprises a wide flange screw to maximize surface area of contact with bone into which the cannulated anchor is inserted. The disclosure provides a stemless implantable device for arthroplasty wherein said flexible connector is attached to bone using a cannulated anchor, wherein said cannulated anchor is constructed from a biocompatible material selected from the group consisting of medical grade plastic, bioactive materials, carbon nanofibers, carbon plates, carbon strips, ceramic, composite materials, hydroxyapatite (HA) coatings, nickel-free super-clastic metal alloys, polyetheretherketone (PEEK), silicone, stainless steel, titanium alloys, titanium, ultra-high molecular weight polyethylene (UHMWPE), ceramic materials, nickel-based superalloys, cobalt-chromium alloys, nitinol alloys, other materials which may promote bone regeneration or materials derived therefrom, and combinations thereof. The disclosure provides a stemless implantable device for arthroplasty wherein said flexible connector is attached to bone using a cannulated anchor, wherein the cannulated anchor has a crystalline surface treatment to promote osteointegration of the anchor within cancellous bone. The disclosure provides a stemless implantable device for arthroplasty wherein said flexible connector is attached to bone using a cannulated anchor, wherein the cannulated anchor has a varying pitch and/or a varying diameter. The disclosure provides a stemless implantable device for arthroplasty wherein said flexible connector is attached to bone using an anchor, wherein the anchor is a cap mechanically fastened to cortical bone to prevent lateral migration. The disclosure provides a stemless implantable device for arthroplasty wherein said flexible connector is attached to bone using an anchor, wherein said anchor comprises an osteointegrated cortical bone cap. The disclosure provides a stemless implantable device for arthroplasty wherein said flexible connector is attached to bone using an anchor, wherein said anchor comprises bone material that is affixed within a cortical divot flush with a surface of cortical bone over the rod head after insertion of said flexible connector into both anchor rods. The disclosure provides a stemless implantable device for arthroplasty wherein said flexible connector is attached to bone using an anchor, wherein said anchor is a temporary fixation of a cortical bone cap using biodegradable material dimensionally larger than the cortical bone cap mechanically fastened to adjacent stable cortical bone to secure the cortical bone cap until osteointegration is achieved. The disclosure provides a stemless implantable device for arthroplasty wherein said flexible connector is attached to bone using an anchor, wherein the anchor is a wide flange cap screwed or locked into the head of an anchor rod and mechanically fastened to cortical bone using screws.

The disclosure provides a stemless implantable device for arthroplasty wherein said proximal fixation system and/or said distal fixation system independently or concurrently is attached to bone using an anchor, wherein the anchor is a flexible connector that is press-fitted directly into bone. The disclosure provides a stemless implantable device for arthroplasty wherein said flexible connector is attached to bone using an anchor, wherein the anchor is a flexible connector comprising expanding hydrogel composite that is press-fitted directly into bone. The disclosure provides a stemless implantable device for arthroplasty wherein said flexible connector is attached to bone using an anchor, wherein the anchor is a flexible connector that is press-fitted directly into bone with expanding hydrogel composite to increase stability as it cures. The disclosure provides a stemless implantable device for arthroplasty wherein said flexible connector is attached to bone using an anchor, wherein the anchor is made of magnetic material. The disclosure provides a stemless implantable device for arthroplasty wherein said flexible connector is attached to bone using an anchor, wherein the anchor has biometric monitoring capabilities. The disclosure provides a stemless implantable device for arthroplasty wherein said flexible connector is attached to bone using an anchor, wherein the anchor comprises bone scaffold integration. The disclosure provides a stemless implantable device for arthroplasty wherein said flexible connector is attached to bone using an anchor, wherein the anchor comprises barbs configured to break away in response to application of a specific controlled mechanical action.

The disclosure provides a stemless implantable device for arthroplasty further comprising a first integrally woven sleeve at the proximal end of said flexible connector to receive and hold a proximal fixation rod, and a second integrally woven sleeve at the distal end of said flexible connector to receive and hold a distal fixation rod. The disclosure provides a stemless implantable device for arthroplasty wherein said first integrally woven sleeve, and second integrally woven sleeve are independently or concurrently barbed. The disclosure provides a stemless implantable device for arthroplasty wherein said proximal fixation rod and/or said distal fixation rod are independently or concurrently barbed. The disclosure provides a stemless implantable device for arthroplasty wherein said first integrally woven sleeve, and second integrally woven sleeve are independently or concurrently smooth. The disclosure provides a stemless implantable device for arthroplasty wherein said proximal fixation rod and/or said distal fixation rod are independently or concurrently smooth. The disclosure provides a stemless implantable device for arthroplasty wherein said proximal fixation rod and/or said distal fixation rod independently or concurrently further comprise an anti-pullout peg. The disclosure provides a stemless implantable device for arthroplasty wherein said flexible connector comprises a fatigue-resistant biocompatible material. The disclosure provides a stemless implantable device for arthroplasty wherein said flexible connector comprises a fatigue-resistant biocompatible material, wherein the fatigue-resistant biocompatible material is selected from the group consisting of medical grade plastic, bioactive materials, carbon nanofibers, carbon plates, carbon strips, composite materials, elastomeric polymers, hydroxyapatite (HA) coatings, nickel-free super-elastic metal alloys, nitinol, internal shape memory alloy, polyetheretherketone (PEEK), silicone, titanium alloys, titanium, ultra-high molecular weight polyethylene (UHMWPE), engineered polymers, ultra-high molecular weight polyethylene, high molecular weight polyethylene, aromatic polyamide, polymers made to be radiopaque, or materials derived therefrom, and combinations thereof. Any of the polymers used to form one or more components of the implantable device could be made radiopaque by the addition of iodine- or bromine-based monomers, or heavy-metal-containing monomers to obtain radiopaque polymer matrices so as to permit tracking of accumulated damage via x-ray, without having to have physical access to the implantable device.

The disclosure provides a stemless implantable device for arthroplasty wherein said flexible connector comprises a fatigue-resistant biocompatible material, wherein the fatigue-resistant biocompatible material is braided nanofiber elements woven in two dimensional and/or three-dimensional arrays. The disclosure provides a stemless implantable device for arthroplasty wherein said flexible connector incorporates ranges of strength, flexibility, elasticity and other material properties so as to optimize the path of motion of patient phalanges and durability of the device. The disclosure provides a stemless implantable device for arthroplasty wherein said flexible connector is constructed from a material configured to remain malleable post-implantation. The disclosure provides a stemless implantable device for arthroplasty further comprising malleable material between anchor and flexible connector. The disclosure provides a stemless implantable device for arthroplasty wherein said flexible connector is in a kit which includes standard various angled implants to correct anchor misalignment. The disclosure provides a stemless implantable device for arthroplasty wherein the flexible connector comprises a flexible material that allows for smooth flexion within a normal range of zero to one hundred (0-100) degrees when activated by a patient's flexor tendon system.

The disclosure provides a stemless implantable device for arthroplasty wherein said flexible connector is a flexible planar component constructed from a fatigue-resistant biocompatible material configured to allow motion of the joint in the sagittal plane when activated by a patient's flexor tendon system. The disclosure provides a stemless implantable device for arthroplasty wherein said flexible connector is a flexible planar component constructed from a fatigue-resistant biocompatible material configured to allow constrained accessory motion when subjected to external forces such as when used to assist in gripping an oddly shaped object. The disclosure provides a stemless implantable device for arthroplasty wherein the width of the flexible connector does not exceed an intracortical dimension measured in the coronal plane of a patient's bone at the site of implantation. The disclosure provides a stemless implantable device for arthroplasty wherein the flexible connector is designed so that the path of motion in the sagittal plane follows an arc path described by a normal interphalangeal joint or approximation thereof. The disclosure provides a stemless implantable device for arthroplasty wherein the flexible connector is designed so that motion in the coronal plane is restricted to a maximum of about 0 degrees of accessory motion. The disclosure provides a stemless implantable device for arthroplasty wherein the flexible connector comprises woven or laminated components to create a flexible mesh with a variety of dynamic qualities and fixation methods. The disclosure provides a stemless implantable device for arthroplasty wherein the flexible connector comprises woven or laminated components to create a flexible mesh with a variety of dynamic qualities and fixation methods and is secured to the bones of the first and second phalange using a fixation system comprised of a rod pocket, or sleeve, wherein the rod pocket or sleeve is integrally woven into the proximal edge and distal edge of said flexible connector.

The disclosure provides a stemless implantable device for arthroplasty wherein the flexible connector maintains proper joint spacing to prevent bone-on-bone contact of articular surfaces of the first and second bones of a joint. The disclosure provides a stemless implantable device for arthroplasty wherein the flexible connector is constructed using a plain weave and using a wide cross-sectional profile for warp components, which run perpendicular to the fixation pins or rods within the implant device, to achieve a spring mesh which confers lateral stability and alignment in the coronal plane without reliance on joint ligaments. The disclosure provides a stemless implantable device for arthroplasty wherein the flexible connector is constructed using a weave pattern that changes in length as tension is applied. The disclosure provides a stemless implantable device for arthroplasty wherein the flexible connector comprises a spring mesh constructed using a bias weave of wide flat sections. The disclosure provides a stemless implantable device for arthroplasty wherein the flexible connector comprises a unitary flexible having a plurality of weave patterns, laminations, or polymer curing formulae. The disclosure provides a stemless implantable device for arthroplasty wherein the flexible connector comprises a fatigue-resistant biocompatible material further comprising a braided nanofiber element. The disclosure provides a stemless implantable device for arthroplasty wherein the flexible connector comprises an internal shape memory alloy. The disclosure provides a stemless implantable device for arthroplasty wherein the flexible connector comprises an internal shape memory alloy which comprises nitinol. The disclosure provides a stemless implantable device for arthroplasty wherein the flexible connector comprises an internal reinforcing textile matrix. The disclosure provides a stemless implantable device for arthroplasty wherein the flexible connector comprises a braided nanofiber element woven in a two-dimensional array. The disclosure provides a stemless implantable device for arthroplasty wherein the flexible connector comprises a braided nanofiber element woven in a three-dimensional array. The disclosure provides a stemless implantable device for arthroplasty wherein the flexible connector comprises a fiber diameter or cross-sectional profile is specified to deliver a Young's modulus of elasticity suitable to the weave pattern so as to allow stretch capacity in flexion of approximately 25% of the unflexed mesh length. The disclosure provides a stemless implantable device for arthroplasty wherein the flexible connector is constructed using engineered polymers. The disclosure provides a stemless implantable device for arthroplasty wherein the flexible connector comprises a polymer configured to permit translation movement and stretch movement that approximates a natural physiologic motion of the interphalangeal joint. The disclosure provides a stemless implantable device for arthroplasty wherein the flexible connector wherein said polymer is silicone. The disclosure provides a stemless implantable device for arthroplasty wherein the flexible connector comprises silicone printed in engineered patterns to improve the flexible connector's response to stresses. The disclosure provides a stemless implantable device for arthroplasty wherein the flexible connector comprises silicone printed in graduated density so that the flexible connector comprises a higher density within or near to the fixation system and a lower density in a different part of the flexible connector where greater flexibility is required to achieve a desired trajectory path of motion. The disclosure provides a stemless implantable device for arthroplasty wherein the flexible connector is a polymer connector which comprises bumper flanges that are oriented in a perpendicular aspect to the coronal plane of the polymer connector to prevent bone-on-bone contact. The disclosure provides a stemless implantable device for arthroplasty wherein the flexible connector is a polymer connector which comprises bumper flanges that are oriented in a perpendicular aspect to the coronal plane of the polymer connector to prevent bone-on-bone contact, further wherein said bumper flanges are gel-filled to allow for dynamic response under various tension and compression conditions.

The disclosure provides a stemless implantable device for arthroplasty wherein the flexible connector is a polymer connector which comprises bumper flanges that are oriented in a perpendicular aspect to the coronal plane of the polymer connector to prevent bone-on-bone contact, further wherein said bumper flange incorporates a hollow portion or gel-filled portion to allow translation of the second bone relative to the first bone along the arc of motion trajectory. The disclosure provides a stemless implantable device for arthroplasty wherein the flexible connector comprises a biocompatible material which is fatigue resistant, and wherein the fatigue-resistant biocompatible material comprises at least two different thicknesses. The disclosure provides a stemless implantable device for arthroplasty wherein the flexible connector comprises a fatigue-resistant biocompatible material comprising at least two different widths. The disclosure provides a stemless implantable device for arthroplasty wherein the flexible connector comprises a fatigue-resistant biocompatible material further comprising a braided nanofiber element. The disclosure provides a stemless implantable device for arthroplasty wherein the flexible connector comprises a fatigue-resistant material that is formed into thickened barrel shape at its proximal and distal edges. The disclosure provides a stemless implantable device for arthroplasty wherein the flexible connector comprises a biocompatible material which is fatigue resistant, and wherein the fatigue-resistant biocompatible material is configured to bend along a prescribed path when stressed by an externally exerted bending force, and further configured to return to a straight position when released from said externally exerted bending force. The disclosure provides a stemless implantable device for arthroplasty wherein the flexible connector comprises a unitary flexible having a plurality of weave patterns, laminations, or polymer curing formulae. The disclosure provides a stemless implantable device for arthroplasty wherein the flexible connector has in-Situ malleability which is adjustable post-implantation. The disclosure provides a stemless implantable device for arthroplasty wherein the flexible connector has inherent adjustment capabilities which can rectify misalignment caused by non-parallel relationship of proximal and distal anchors. The disclosure provides a stemless implantable device for arthroplasty wherein the flexible connector comprises malleable material between the anchor and the flexible connector. The disclosure provides a stemless implantable device for arthroplasty wherein the flexible connector is malleable due to variable density, curing, material properties, construction, geometry, manufacturing processes, and/or other factors. The disclosure provides a stemless implantable device for arthroplasty wherein the flexible connector comprises a beam with no bumper; may impart stenting behavior to keep bone surfaces apart from each other, may have a memory to return to a pre-flexed neutral angle of flexion; and/or may have a graduated or variable density with variable characteristics of strength and flexibility. The disclosure provides a stemless implantable device for arthroplasty wherein the flexible connector is in a kit which comprises a plurality of standard various angled implants to correct anchor misalignment. The disclosure provides a stemless implantable device for arthroplasty wherein the flexible connector is in a kit which comprises a plurality of flexible connectors with various pre-flexed neutral angles of flexion.

The disclosure provides a system for affixing a rod to a flexible connector comprising a hollow cylinder with an arc opening of approximately 0.2 radians and with flanges substantially parallel to one another protruding from the edges of the rod, the flanges being configured to secure a flexible connector within the sectional profile of the hollow cylinder. The disclosure provides a system for affixing a flexible connector comprising a cannulated screw anchor with longitudinal slot with an arc opening of approximately 0.2 radians.

The disclosure provides a surgical procedure for implanting a stemless implantable device for arthroplasty, comprising selecting a patient in need of implantation of a stemless implantable device for arthroplasty; providing a stemless implantable device for arthroplasty as disclosed herein, and implanting the stemless implantable device for arthroplasty into said patient. The disclosure provides a surgical procedure for implanting a stemless implantable device for arthroplasty comprising the steps of: performing a minimally invasive lateral incision at an implant site of a patient; preparing a joint capsule and one or more phalangeal bones at the implant site; press-fitting a lateral insertion of the stemless implantable device for arthroplasty into the implant site or into a pre-fitted anchor system; and performing a surgical closure of a wound at the implant site. The disclosure provides a surgical procedure for implanting a stemless implantable device for arthroplasty wherein preparing the joint capsule and phalangeal bones includes resection of damaged cartilage and articular surfaces. The disclosure provides a surgical procedure for implanting a stemless implantable device for arthroplasty further comprising capping countersunk anchor holes using resected bone.

In accordance with yet another embodiment, the present disclosure provides a use of the devices and methods as described herein, and at least one additional therapeutic agent or modality, for use in treating a disease or disorder, for example, as set forth herein, in a patient.

Embodiments of the present invention also provide a method for treating and/or preventing a disease or condition as set forth herein in a patient, wherein said method comprises: selecting a patient in need of treating and/or preventing said disease or condition as set forth herein; administering to the patient the method(s) and/or device(s) of the disclosure, thereby treating and/or preventing said disease in said patient.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—depicts a diagram of a typical human hand skeleton for reference to standard medical nomenclature used to describe various anatomical elements.

FIG. 2—depicts a perspective illustration of a human hand indicating standard nomenclature used to describe spatial relationships between elements.

FIG. 3—depicts a side view of the interphalangeal bones of a human finger shown in a naturally flexed position superimposed in planar alignment with an image of the bones of the same finger shown in an extended position and partially flexed position indicating the arc of motion of the second phalange relative to the first phalange. The flexed position is depicted with solid lines, the extended position is depicted with dashed or dotted; solid line traces the arc of motion about an axis of rotation located within the first phalange.

FIG. 4—shows a perspective view of the hand with index finger in an extended position opposed by an axial force imposed by the thumb of the same hand indicating one type of accessory force resisted by the interphalangeal joint in ordinary use. This is referred to as a “key pinch”.

FIG. 5—depicts an illustration of a conceptual embodiment of the device as disclosed herein showing the first fixation rod, mesh hinge and second fixation rod.

FIG. 6—depicts a perspective view of an exemplary embodiment of the holistic device showing a proximal cannulated wide flange threaded anchor with radial slot and reverse-threaded anchor cap and a parallel distal connector of similar dimension and configuration aligned in a mirrored relationship about the medial axis of one embodiment of a flexible connector with dorsal and volar bumpers and a continuous fixation boss at the proximal edge and distal edge of said flexible connector inserted into the proximal and distal anchors respectively and secured therein by one embodiment of an anchor cap screwed into the head of each anchor.

FIG. 7—depicts a cross sectional view of an exemplary embodiment of the holistic device as disclosed herein in the extended (unflexed) position showing dorsal and volar bumper flanges and thickened barrels at proximal and distal edges and keyhole slot at the proximal condyle head.

FIG. 8—depicts a planar view of an exemplary embodiment of the holistic device as disclosed herein in the extended position viewed from the dorsal aspect.

FIG. 9—depicts an exploded perspective view of an exemplary embodiment of the holistic device as disclosed herein in the extended position.

FIG. 10—depicts a side view of one embodiment of the holistic device as disclosed herein in the anatomy extended position.

FIG. 11—side view of an exemplary embodiment of the holistic device as disclosed herein in the anatomy in a flexed position.

FIG. 12—perspective view of an exemplary embodiment of the holistic device as disclosed herein in the anatomy in a partially flexed position.

FIG. 13—is a cross sectional view of one embodiment of the holistic device as disclosed herein in the anatomy extended position.

FIG. 14—Perspective view of an exemplary embodiment of the holistic device as disclosed herein utilizing a magnetized component in the proximal transverse anchor in roughly parallel relationship with a magnetized component in the distal transverse anchor possessing the same magnetic polarity so as to exert a stenting force between the proximal and distal magnetized components.

FIG. 15—Perspective view of an exemplary embodiment of the holistic device as disclosed herein utilizing a magnetized component in the proximal transverse anchor in roughly parallel relationship with a magnetized component in the distal transverse anchor possessing an attractive magnetic polarity so as to exert a stabilizing force between the proximal and distal magnetized components.

FIG. 16—depicts a perspective view of one embodiment of a fixation anchor showing engagement barbs.

FIG. 17—side view of an exemplary embodiment of cannulated threaded anchor.

FIG. 18—head end view of cannulated threaded anchor.

FIG. 19—perspective view of one embodiment of a threaded screw cap

FIG. 20—exploded perspective view depicting an exemplary embodiment of a magnetic cap system configured to confer a stenting force while containing a connector within the anchor.

FIG. 21—(A) exploded perspective view depicting an exemplary embodiment of a holistic device as disclosed herein showing a cannulated anchor before and after insertion of the connector. (B) exploded perspective view depicting an exemplary embodiment of a reverse-threaded screw cap affixed to the head of cannulated anchor after insertion of the connector.

FIG. 22—cross section of one embodiment of a flexible connector comprised of a hydrophilic coating and indicating the expansion zone of said coating, and further showing a guide dot. The hydrophilic coating expands within a cannulated anchor or prepared hole in bone.

FIG. 23—depicts a longitudinal section through one embodiment of a mesh hinge illustrating an integrated rod pocket, or sleeve, woven into the connector to receive the proximal rod and distal rod which are inserted into the respective proximal and distal rod sleeves.

FIG. 24—depicts a cross sectional view of one embodiment of the holistic device as disclosed herein in the anatomy extended position.

FIG. 25—depicts a cross sectional view of one embodiment of the holistic device as disclosed herein showing 3D woven fabric with lozenge shaped weft fiber profile and collapsible cage warp structure resulting in lengthening of the fabric along the sagittal axis when flexed and preservation of coronal stability conferred by the 3D warp structure.

FIG. 26—depicts a perspective view of one embodiment of a connector fabric showing core elements comprised of a plurality of thin flat longitudinal strips of titanium or similar biocompatible material spaced evenly across the width of the connector and woven together by carbon nanofibers or carbon nanofiber braids or similar fatigue resistant flexible material in a weave pattern comprising variable cross-sectional profiles for warp and weft fibers to customize stiffness and stability.

FIG. 27—depicts a cross sectional view of one embodiment of a flexible connector with hollow bumpers.

FIG. 28—depicts a flow diagram of the detail components of the bone preparation phase of the surgical procedure: (A) minimally invasive surgical access via a mid-lateral incision; (B) guidewire insertion into a phalangeal bone; (C) external jig alignment with guidewires; (D) a cannulated drill bit, drilling into a phalangeal bone along a guidewire; (E) A sagittal saw used to remove a wedge of bone; (F) A sagittal saw used to remove a wedge of bone.

FIG. 29—depicts guidewire insertion into a phalangeal bone.

FIG. 30—depicts a cannulated drill bit, drilling into a phalangeal bone along a guidewire.

FIG. 31—depicts an external alignment jig next to phalangeal bones.

FIG. 32—(A) is a perspective view of a smooth fixation rod with a rotatable end cap; (B) is a perspective view of a smooth fixation rod with a cuff-link style end cap.

FIG. 33—is a perspective view of (A) a smooth fixation rod with fluting; (B) a smooth fixation rod with surface treated to allow for osseointegration.

FIG. 34A—is a perspective view of an example stemless implantable device for arthroplasty that includes a mesh hinge.

FIG. 34B—is a side view of the stemless implantable device for arthroplasty of FIG. 34A.

FIG. 35A—is a perspective view of another example stemless implantable device for arthroplasty.

FIG. 35B—is a side view of the stemless implantable device for arthroplasty of FIG. 35A.

FIG. 36—is a top-down view of an example flexible connector.

FIG. 37—is a top-down view of another example flexible connector.

FIG. 38—is a perspective view of another example flexible connector.

FIG. 39—is a perspective view of an example fixation rod.

FIG. 40—is a perspective view of another example fixation rod.

FIG. 41—is a perspective view of another example fixation rod.

FIG. 42A—is a perspective view of another example stemless implantable device for arthroplasty.

FIG. 42B—is a side view of the example stemless implantable device for arthroplasty of FIG. 42A.

FIG. 43—is a perspective view of another example stemless implantable device for arthroplasty.

FIG. 44—is a perspective view of another example stemless implantable device for arthroplasty.

FIG. 45—is a perspective view of another example stemless implantable device for arthroplasty.

### DETAILED DESCRIPTION OF EXEMPLARY EMBODIMENTS

An amount is “effective” as used herein, when the amount provides an effect in the subject. As used herein, the term “effective” means an amount of a compound or composition, or device(s) and method(s), sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective therapy, such as a compound or composition, or device(s) and method(s), as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic methods, devices, or agents).

As used herein, the terms “therapies” and “therapy” can refer to any method(s), device(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “about” when used in conjunction with a stated numerical value or range has the meaning reasonably ascribed to it by a person skilled in the art, i.e., denoting somewhat more or somewhat less than the stated value or range.

As used herein, the phrase “at least one of” followed by a list of alternatives is to be interpreted as encompassing any single one of the alternatives or any combination of multiple of the alternatives. For example, “at least one of A, B, or C” includes A alone, B alone, C alone, A and B in combination, A and C in combination, B and C in combination, and a combination of A, B, and C.

### Stemless Implantable Device

In general, embodiments of the stemless implantable device of the present disclosure comprises a flexible connector that spans the joint space between, for example, the first phalanx and the second phalanx in the coronal plane and flexes in the sagittal plane, to serve as a joint replacement system. The devices and methods of the present disclosure are also particularly suited to treating synovial joints such as the small joints of the hand, wrist, elbow, shoulder, ankle, foot, jaw, and spine and in some cases may be suitable for use in large joints of the hip and knee. In case of traumatic injury the devices and methods may be used to provide articulation in lieu of a damaged or amputated joint. In certain embodiments, the flexible connector is secured along its proximal edge by, for example, a fixation system implanted transversely in the coronal plane through the first phalange and along its distal edge by a similar fixation system implanted transversely through the second phalange in parallel relationship with the first fixation system. In exemplary embodiments, the overall dimensions of the joint replacement system may be confined within an envelope bounded by the superficial cortical surfaces of the bones of the original joint or of a similar joint in the patient or subject of similar size as the patient. In certain embodiments of the disclosure, custom implants can be designed for various patient profiles, e.g., athlete, musician, or laborer. These can have differential material strengths for various performance specifications.

In certain embodiments as disclosed herein, the stemless implantable device of the present disclosure and its components can be made of a biocompatible material. A biocompatible material is to be understood as being a material with low level of immune response. Biocompatible materials are sometimes also referred to as biomaterials. Analogous are biocompatible metals, a metal with low immune response such as titanium or tantalum. The biocompatible metal could also be a biocompatible alloy comprising at least one biocompatible metal. In certain embodiments, the biocompatible material may be, for example, formed of any suitable medical grade material, such as biocompatible metals such as stainless steel, titanium, titanium alloys, etc. or a medical grade plastic, such as polyetheretherketone (PEEK), or ceramic, or another radiolucent material, ultra-high molecular weight polyethylene (UHMWPE), etc. In certain embodiments, the biocompatible material may be, for example, stainless steel, titanium, titanium alloys, a medical grade plastic, silicone, polyetheretherketone (PEEK), ceramic, ultra-high molecular weight polyethylene (UHMWPE), carbon nanofibers, carbon strips, carbon plates, ceramic materials, nickel-based superalloys, cobalt-chromium alloys, nitinol alloys, and combinations thereof. If so desired, the implant may also be formed of a bioresorbable material. The bioresorbable material may be osteoconductive or osteoinductive (or both).

The implantable medical device according to any of the embodiments disclosed herein, including any components thereof, could comprise at least one material selected from a group consisting of: polytetrafluoroethylene (PTFE), perfluoroalkoxy (PFA) and fluorinated ethylene propylene (FEP). It is furthermore conceivable that the material comprises a metal alloy, such as cobalt-chromium-molybdenum or titanium or stainless steel, or polyethylene, such as cross-linked polyethylene or gas sterilized polyethylene. The use of ceramic material is also conceivable, in the contacting surfaces or the entire medical device such as zirconium ceramics or alumina ceramics. The part of the medical device in contact with human bone for fixation of the medical device to human bone could comprise a porous structure which could be a porous micro or nano-structure adapted to promote the growth-in of human bone in the medical device for fixating the medical device. The porous structure could be achieved by applying a hydroxyapatite (HA) coating, or a rough open-pored titanium coating, which could be produced by air plasma spraying, a combination comprising a rough open-pored titanium coating and a HA top layer is also conceivable. The contacting parts could be made of a self-lubricated material such as a waxy polymer, such as PTFE, PFA, FEP, PE and UHMWPE, or a powder metallurgy material which could be infused with a lubricant, which preferably is a biocompatible lubricant such as a Hyaluronic acid derivate. In certain embodiments as disclosed herein the material of contacting parts or surfaces of the implantable medical device herein is adapted to be constantly or intermittently lubricated. According to some embodiments the parts or portions of the medical device could comprise a combination of metal materials and/or carbon fibers and/or boron, a combination of metal and plastic materials, a combination of metal and carbon-based material, a combination of carbon and plastic based material, a combination of flexible and stiff materials, a combination of elastic and less elastic materials, Corian or acrylic polymers.

According to some embodiments the parts or portions of the medical device could comprise a magnetic construction. There are normal forces on the bearing surfaces of a joint. Reducing these normal forces can reduce the load, and therefore the wear of the joint. It is suggested herein that, for example, opposing magnets placed in or on the joints could be used to reduce these normal forces. Since it is desirable for this force reduction to occur it is preferable that this magnetic opposition occurs while the joint flexes or extends. Attracting magnets could also be used to augment implant stability.

The following options include both means to preserve and/or replace the bearing surfaces of the joint. In the certain embodiments as disclosed herein all or part of the components which are anchored to the bone are typically metal and could include and/or be constructed from or include magnetic materials. For example, rare earth magnets could be used with both components having like poles (e.g., negative) facing each other. If it is desired to unload the joint while preserving the bearing surfaces of the joint, the mechanism as shown in FIG. 14 can be utilized.

Opposing magnets in the joint, for example as opposing pairs, can be straight or curved depending upon clinical requirements. Though the opposing magnets are intended to provide a reduction in the normal forces, geometric relationships can be selected to include lateral force vectors to help stabilize the joint. It is possible that these effects could be externally controlled by the application of external magnetic field or be intrinsic properties of the materials.

It is understood that lateral forces can be used to stabilize a joint. These forces can, by their orientation, help to align the path of the elongation and flexion of the joint. The device as disclosed herein can be adjusted relative to each other for proper tracking. They can be angled to the left or right from the natural axis of the relative bending of the joint. In certain clinical situations, it may be desirable to change this relative angle. The gentle magnetic bias imposed by these off axis magnets can result in a reorientation of the relative bending angle. In another embodiment the path of alignment may be adjusted by insertion of a fixed alignment-correcting connector fabricated with non-parallel fixation edges and selected by the surgeon to rectify a specific deviation in alignment of previously implanted transverse anchors.

In other clinical situations, it may be desirable to adjust the radial-ulnar (inside-outside) angle of the joint. Use of magnets on one or both sides of the joint would result in biasing forces which could result in realignment of the side-to-side tracking of the joint, for example to stabilize the joint in situations where the ligaments are not optimal.

According to some embodiments the parts or portions of the medical device could comprise biometric monitoring capabilities. The biometric monitoring as disclosed herein can comprise a sensor system which can be embedded in a thin, adhesive, conforming material that is applied to specific areas of concern. Exemplary areas include the fingers, hips, and knees. These sensors map out the anatomic area. If threshold parameters are exceeded, the sensors inform, for example, a telemetric receiver that, in turn, activates an alarm to a nurse or other health care professional.

Embedded sensors are needed to detect certain internal parameters that are not directly visible to the human eye. These sensors can be used in specific locations to detect specific parameters.

One way of embedding a biometric monitoring sensor is through an open surgical procedure. During such a surgical procedure, the sensor is embedded by a surgeon directly into bone or soft tissue or is attached directly to a secured implant (e.g., arthroplasty in a finger or knee). The sensor system may be used during the surgical procedure to inform the surgeon on the position and/or function of the implant and of soft tissue balance and/or alignment. The sensor is directly embedded with a penetrating instrument that releases the sensor at a predetermined depth. The sensor may be attached to the secured implant with a specific locking system or adhesive. The sensor is activated prior to closure for validating the sensor.

The parameters to be evaluated and time factors determine the energy source required for the embedded sensor. Short time frames (up to 5 years) allow the use of a battery. Longer duration needs suggest use of external activation or powering systems or the use of the patient's kinetic energy to supply energy to the sensor system. These activation systems can be presently utilized. The sensors can also be activated at predetermined times to monitor implant cycles, abnormal motion and implant wear thresholds.

In exemplary embodiments, information may be received telemetrically. In one exemplary embodiment, the sensors are preprogrammed to “activate” and send required information if a specific threshold is exceeded. The sensors could also be activated and used to relay information to an external receiver. Further applications allow readjustment of a “smart implant” to release specific medications, biologics, or other substances, or to readjust alignment or modularity of the implant.

The biometric sensor system may be initially activated and read in a doctor's office and further activation can occur in the patient's house, with the patient having ability to send the information through Internet applications, for example, to the physician. Software may be programmed to receive the information, process it, and then, relay it to the healthcare provider.

The biometric monitoring sensor system as disclosed herein can be used to evaluate function of internal implants. Present knowledge of actual implant function is poor. Physicians continue to use external methods, including X-rays, bone scans, and patient evaluation. However, they are typically left only with open surgical exploration for actual investigation of function. Using biometric monitoring sensor system as disclosed herein permits detection of an implant's early malfunction and impending catastrophic failure. As such, early intervention is made possible. This, in turn, decreases a patient's morbidity, decreases future medical care cost, and increases the patient's quality of life.

The biometric monitoring sensor system as disclosed herein can monitor important parameters of the implant-host system. Exemplary parameters that could be measured include: implant stability, implant motion, implant wear, implant cycle times, implant identification, implant pressure/load, implant integration, joint fluid analysis, articulating surfaces information, ligament function, and many more.

Application of biometric monitoring sensor system as disclosed herein allows one to determine if the implant is unstable and/or if excessive motion or subsidence occurs. In an exemplary application, the sensor can be configured to release an active agent from an activated implanted module to increase integration. Alternatively, and/or additionally, the implant biometric monitoring sensor system as disclosed herein can be used to adjust the angle/offset/soft tissue tension to stabilize the implant if needed.

A joint implant biometric monitoring sensor system as disclosed herein can detect an increase in heat, acid, or other physical property. Such knowledge would provide the physician with an early infection warning. In an exemplary infection treatment application, the sensor can activate an embedded module that releases, for example, an antibiotic.

### Fixation System

As set forth above, certain embodiments of the stemless implantable device of the present disclosure comprise a flexible connector that spans the joint space between, for example, the proximal phalanx and middle phalanx in the coronal plane and flexes in the sagittal plane, to serve as a joint replacement system. In certain embodiments, the flexible connector is secured along its proximal edge by, for example, a fixation system implanted transversely in the coronal plane through the first phalange and along its distal edge by a fixation system implanted transversely through the second phalange in parallel relationship with the first fixation system. In certain embodiments as disclosed herein, the fixation system is a transverse fixation system. In certain embodiments as disclosed herein the fixation system implanted transversely in the coronal plane through the first phalange and along its distal edge by a fixation system implanted transversely through the second phalange are similar. In other embodiments as disclosed herein the fixation system implanted transversely in the coronal plane through the first phalange and along its distal edge by a fixation system implanted transversely through the second phalange are dissimilar.

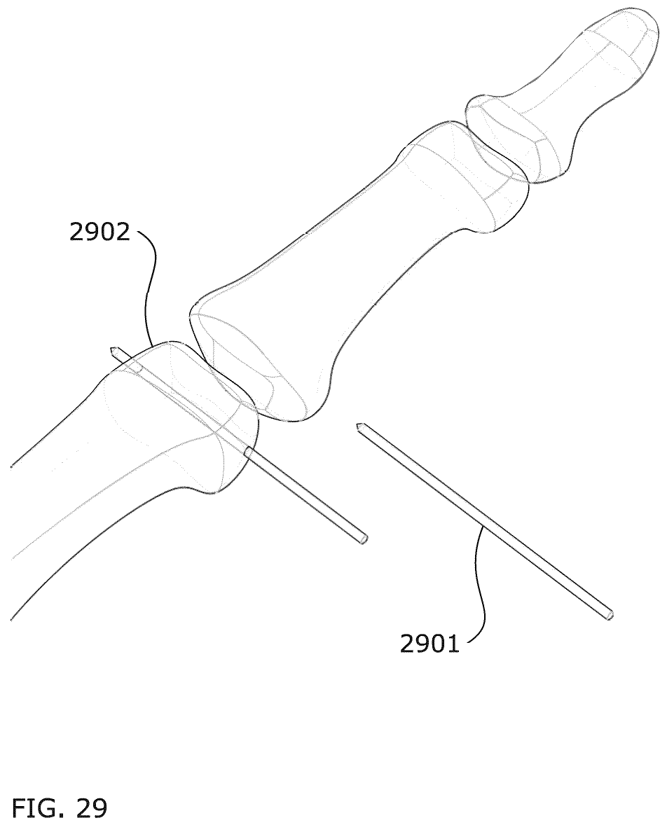

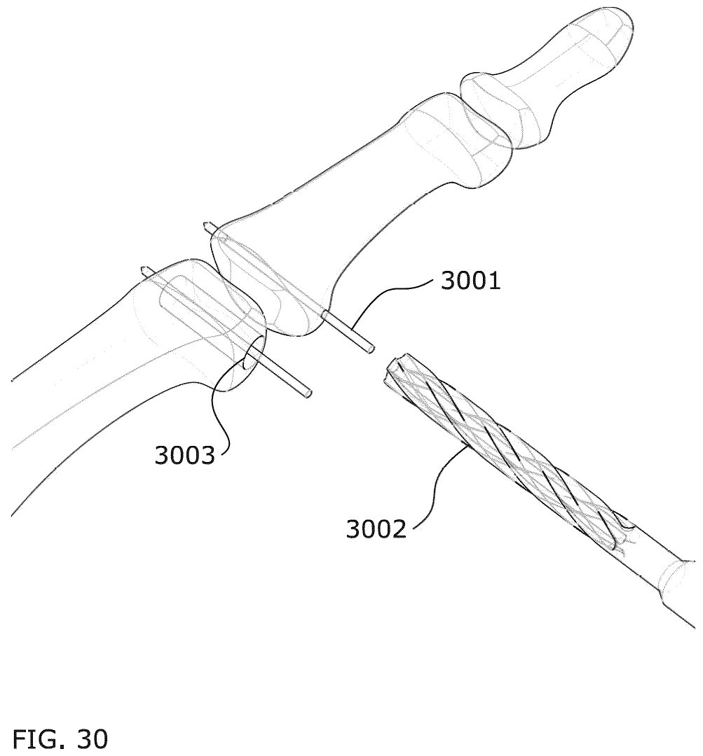

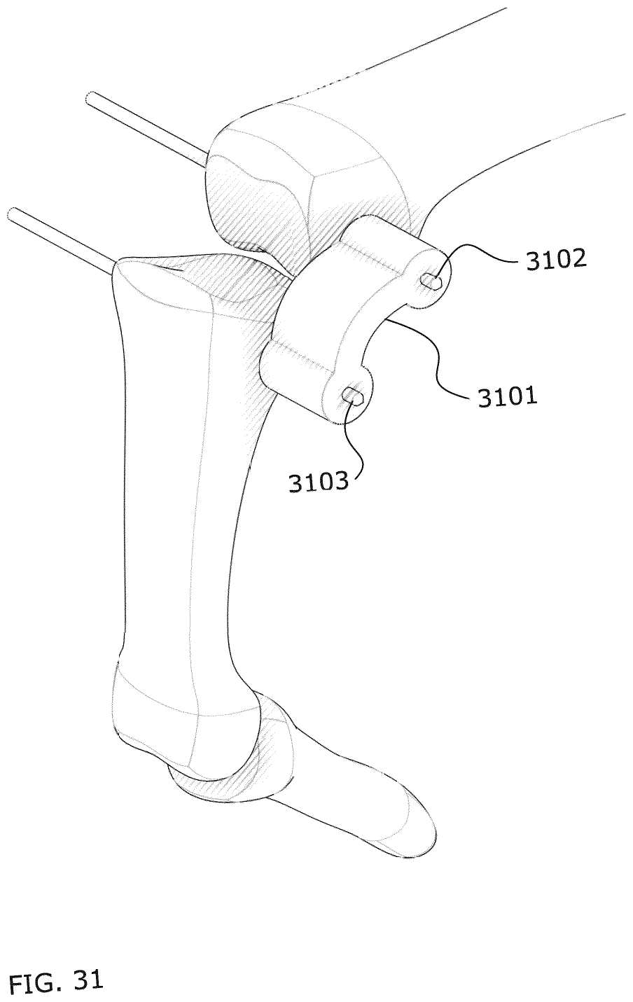

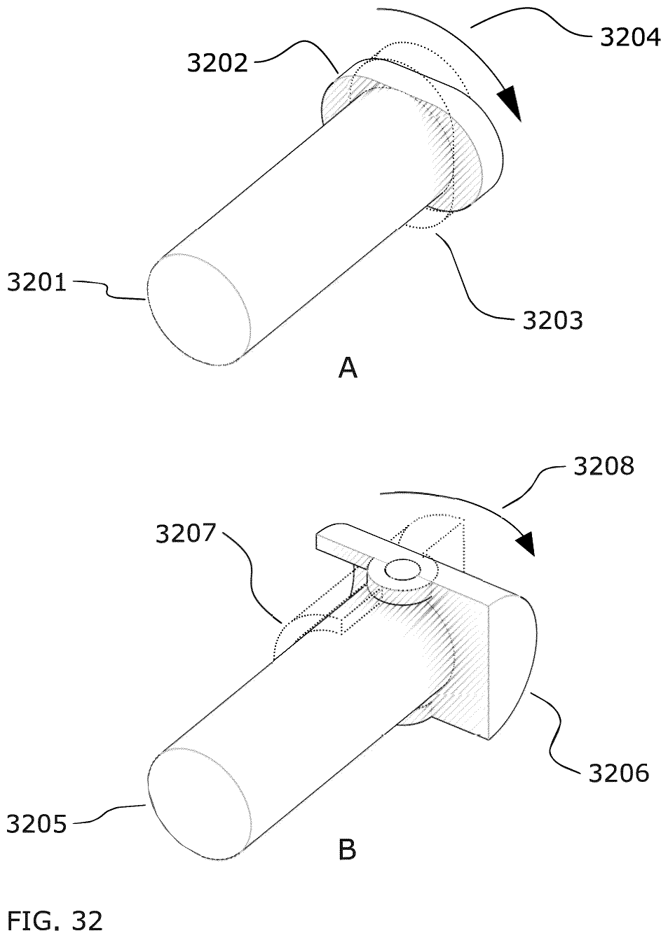

In certain embodiments, the fixation system, such as a transverse fixation system, is made of, for example, a biocompatible material with suitable high fatigue strength and modulus of elasticity similar to bone and surface chemistry that promotes a histologically stable condition at the implant-tissue interface. (See FIG. 32 showing a perspective diagram of smooth anchor rod with a rotatable end cap).

In certain embodiments, the fixation system, such as a transverse fixation system, is made of, for example, a material such as, but not limited to titanium, titanium alloys, composite materials, nickel-free super-clastic metal alloys, UHMWPE, hydroxyapatite (HA) coatings, bioactive materials and composites to promote bone regeneration. (See FIG. 32 for a perspective view of cuff-link head, showing it in operation).

In certain embodiments, the proximal edge of the aforementioned flexible connector may be affixed within the first bone and the distal edge of said proximal anchor may be affixed within the second bone using any fixation method or combination of fixation methods and fixation features such as cemented, uncemented, osteointegration, press fit, screw thread, capped, or other locking system.

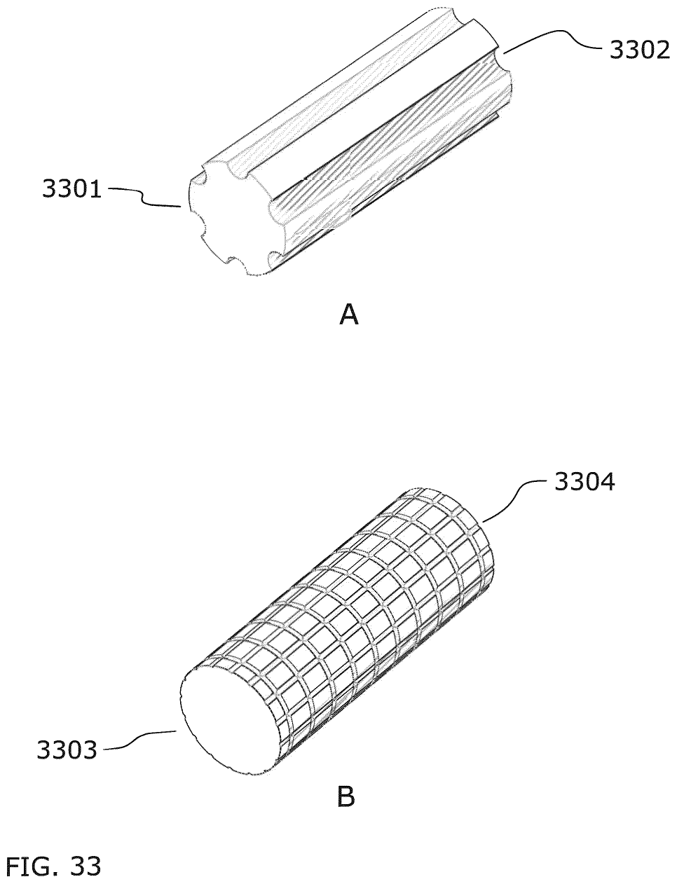

In certain embodiments, the fixation system, such as a transverse fixation system, may comprise surface patterning to enhance natural bone ingrowth to anchor. (See FIG. 33 view showing example of surface patterning; Also see U.S. Pat. No. 10,610,347; U.S. Patent Application Publication No. 2020/0149145; U.S. Patent Application Publication No. 2019/0231511).

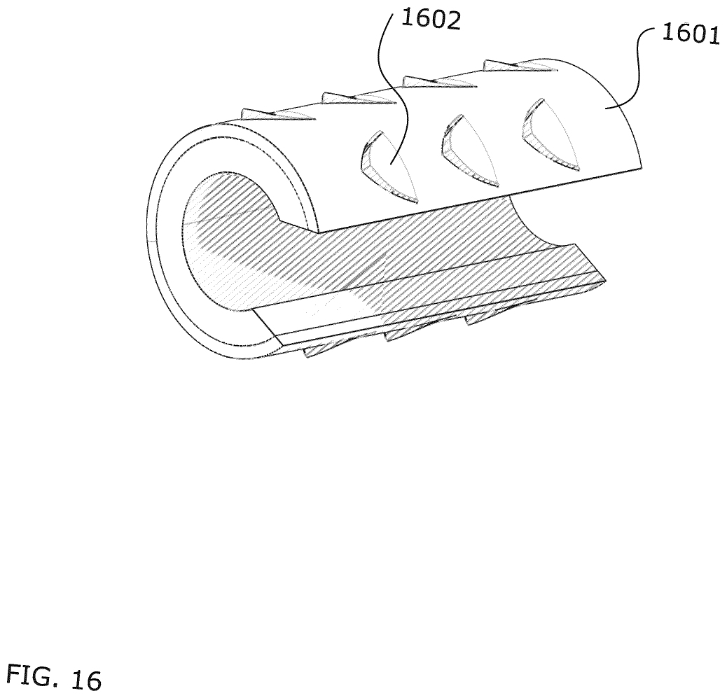

In certain embodiments, the fixation system, such as a transverse fixation system, may comprise multitude of individual “shark's tooth” barbs of sufficient quantity and placement to confer stability within the patient's cancellous bone. The barbs may be configured and manufactured to break away under specific controlled mechanical action to allow expansion of the anchor. (See FIG. 16).

In certain embodiments, the fixation system, such as a transverse fixation system, may comprise concentric fluting pitched in the direction of insertion to prevent dislocation in the direction of extraction.

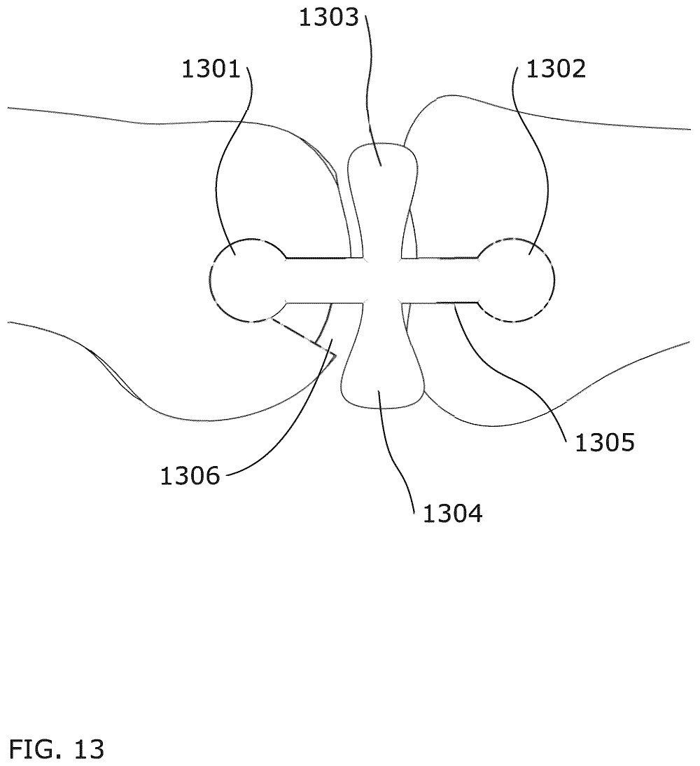

The fixation system for a connector, such as a flexible connector as disclosed herein, in lieu of a rigid pin or rod, may utilize a thickened barrel shape at the proximal and distal edges of said connector to be held fast within the hollow shaft of a cannulated anchor that incorporates a slot of sufficient width to allow the connector to pass through the slot and into the joint cavity. (See FIG. 13).

The fixation system for a connector, such as a flexible connector as disclosed herein, in certain embodiments, has a maximum length which does not exceed the intracortical dimension measured in the coronal plane of the patient's bone at the site of implantation.

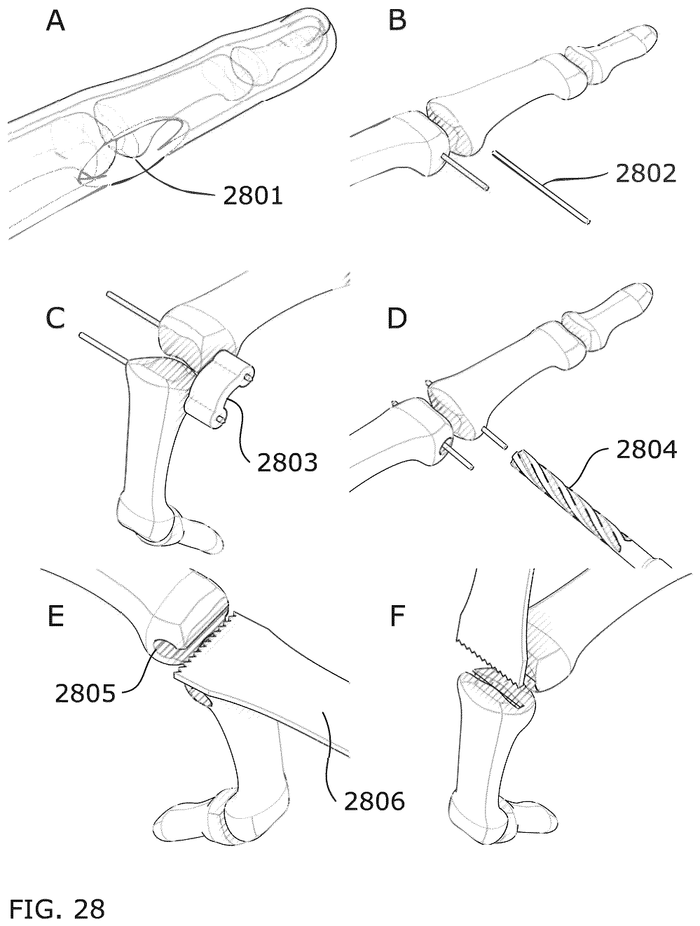

The fixation system for a connector, such as a flexible connector as disclosed herein, in certain embodiments has a maximum dimension measured in the sagittal plane not to exceed, for example, approximately 70% of bone dimension measured in the sagittal plane at the site of implantation after sectioning or debridement, so as to allow sufficient residual bone to prevent bone fracture under normal force loading. (See FIG. 28)

The fixation system may further comprise a rigid rod or pin that may be inserted into the pocket or sleeve to confer stability to said edge so as to facilitate insertion into the prepared bone. (See FIG. 28).

In some embodiments, the rigid rod or pin may be barbed to ensure continuous engagement of the mesh along the full length of the rod or pin and to prevent dislocation of the mesh connector during insertion of the implant device.

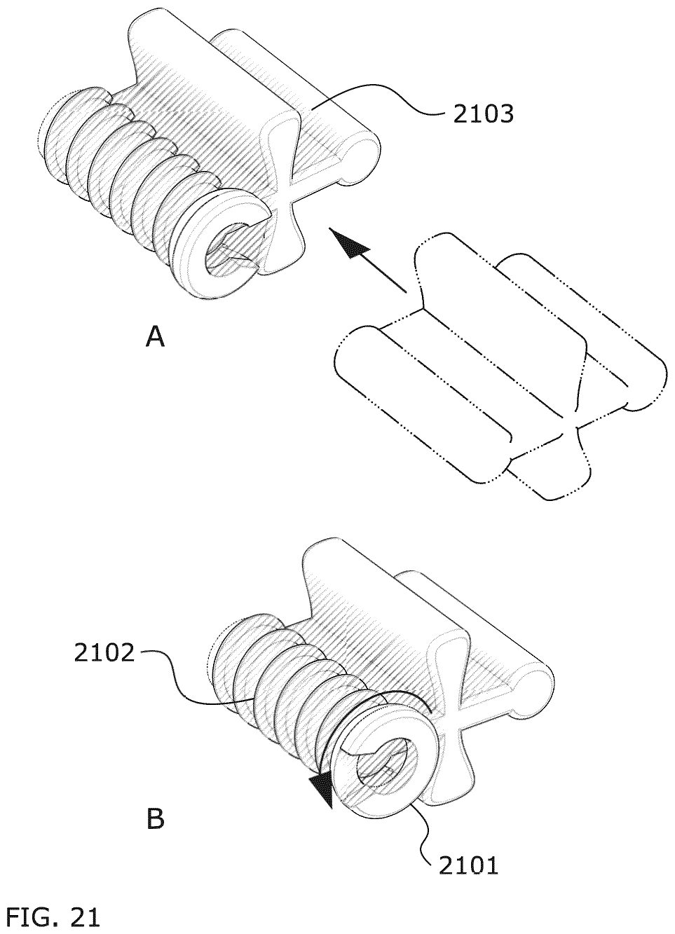

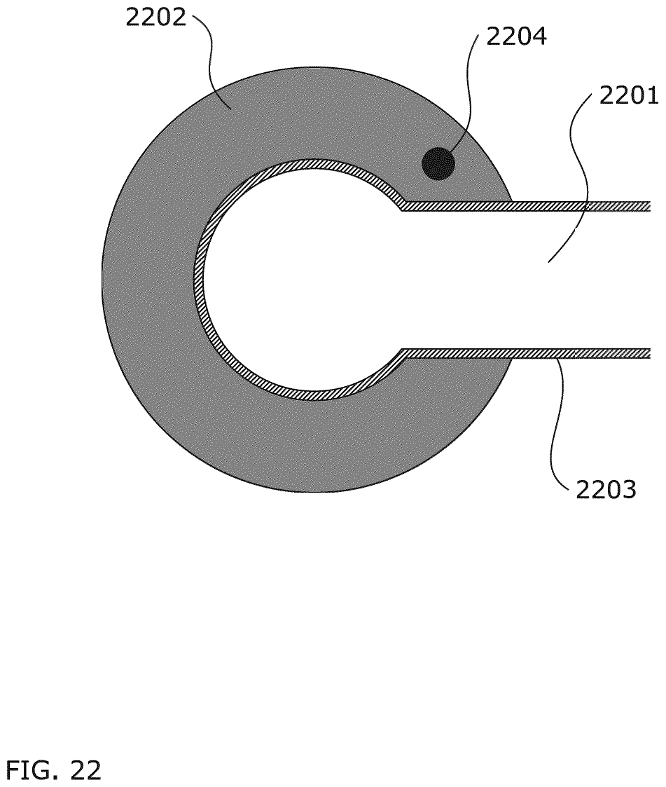

In other embodiments, in lieu of an integrally woven sleeve and barbed rod or pin, a smooth hollow cylinder with crimping flanges may be secured along the proximal and distal edges of the mesh connector. Barbs on the inside surface of the crimping flanges may be employed to further secure connector edges within the hollow cylinder when the crimping flanges are pinched together by a continuous brake. (See FIG. 22).

### Anchor

As set forth above, in certain embodiments the stemless implantable device of the present disclosure comprises a flexible connector that spans the joint space between, for example, the first phalange and the second phalange in the coronal plane and flexes in the sagittal plane, to serve as a joint replacement system. In certain embodiments, the flexible connector is secured along its proximal edge by, for example, a fixation system implanted transversely in the coronal plane through the first phalange and along its distal edge by a fixation system implanted transversely through the second phalange in parallel relationship with the first fixation system. In certain embodiments as disclosed herein, the fixation system is a transverse fixation system. In certain embodiments as disclosed herein, the fixation system for a connector, such as a flexible connector as disclosed herein, is attached to bone using an anchor. In certain embodiments, the anchor is a cannulated anchor. In certain embodiments, the cannulated anchor may assume the configuration of a wide flange screw to maximize surface area of contact with bone into which it is inserted. The cannulated anchor may be manufactured of a material such as titanium or other biocompatible materials as disclosed herein and may optionally have a crystalline surface treatment to promote osteointegration of the anchor within, for example, cancellous bone.

In certain embodiments as disclosed herein the anchor is the device directly in contact with the bone. In certain embodiments as disclosed herein the flexible connector is placed within the anchor. In certain embodiments as disclosed herein there are caps/rivets which are part of the anchor device but are not, by themselves, the anchor proper.

In certain embodiments, the proximal fixation system and/or said distal fixation system is attached to bone using a cannulated anchor, wherein the cannulated anchor has a varying, non-uniform pitch and/or a varying, non-uniform diameter.

In certain embodiments, the anchor is a cap mechanically fastened to cortical bone to prevent lateral migration.

In certain embodiments, the anchor is an osteointegrated cortical bone cap, wherein the patient's bone material salvaged during surgical preparation is affixed within the cortical divot flush with surface of cortical bone over the rod head after insertion of the flexible connector into both anchor rods.

In certain embodiments, the anchor is a temporary fixation of the cortical bone cap using biodegradable material dimensionally larger than cortical bone cap mechanically fastened to adjacent stable cortical bone to secure cortical bone cap until osteointegration is achieved.

In certain embodiments, the anchor is a wide flange cap screwed or locked into the head of the anchor rod and mechanically fastened to cortical bone using screws.

In certain embodiments, the anchor is a flexible connector press-fit directly into bone with no anchor rod or pin fastening, or with expanding hydrogel composite. (See FIG. 13).

In certain embodiments, the anchor comprises a magnetic construction, as set forth herein.

In certain embodiments, the anchor has biometric monitoring capabilities, as set forth herein. In certain embodiments, the anchor has bone scaffold integration.

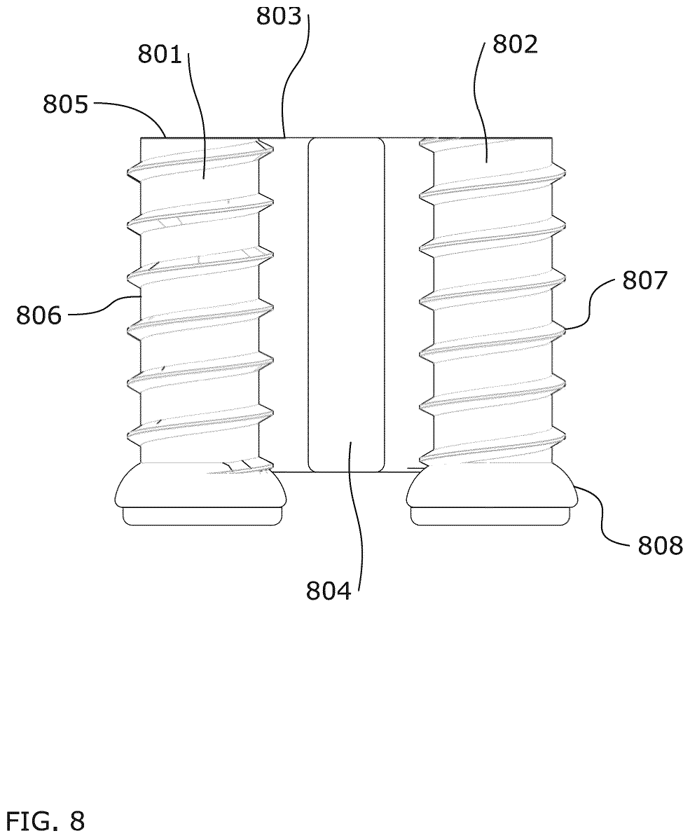

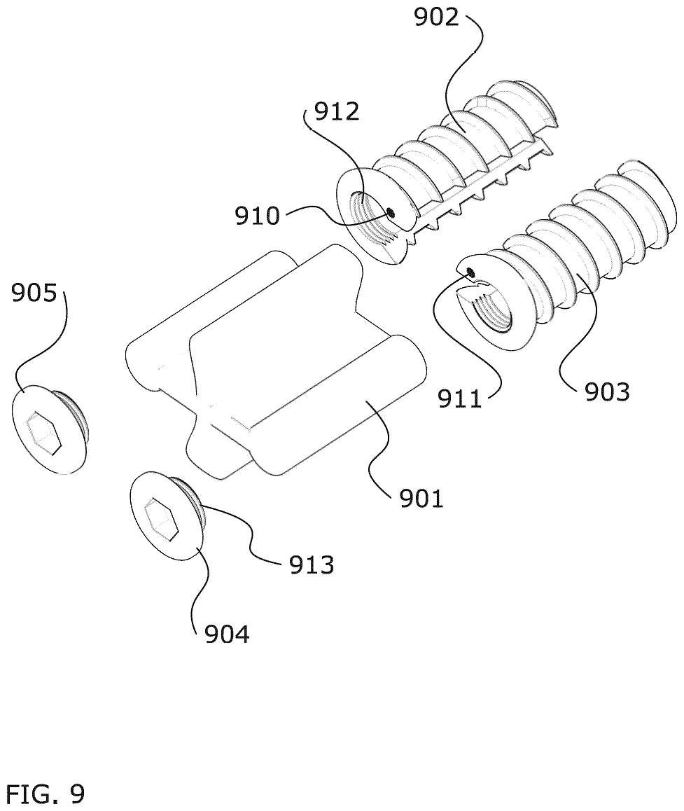

In a preferred embodiment, the anchor has a flute alignment which allows for about 110 degrees of rotation. (See FIG. 33). In certain embodiments as disclosed herein, the anchor has wide thread with fine pitch. In certain embodiments as disclosed herein, the anchor is a cannulated screw with longitudinal slot. In certain embodiments as disclosed herein, the anchor has barbs configured and manufactured to break away under specific controlled mechanical action. (See FIG. 9).

In certain embodiments as disclosed herein, the anchor has endo button-cufflink. (See FIG. 32).

In certain embodiments as disclosed herein, the anchor is a headless or headed screw.

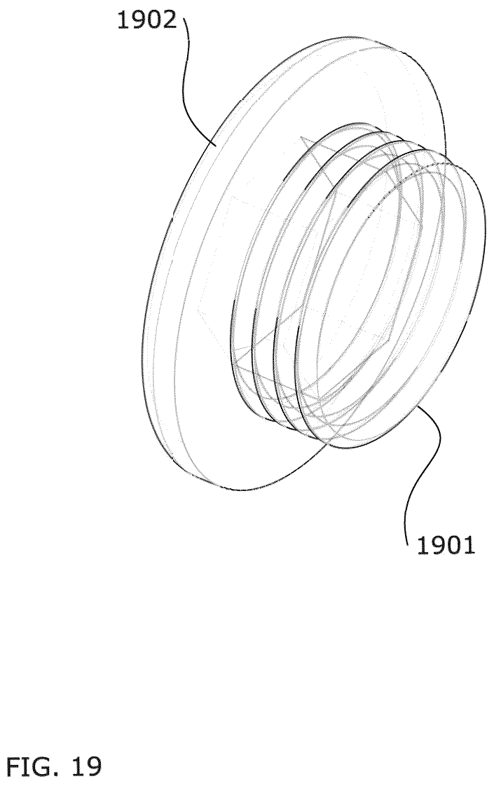

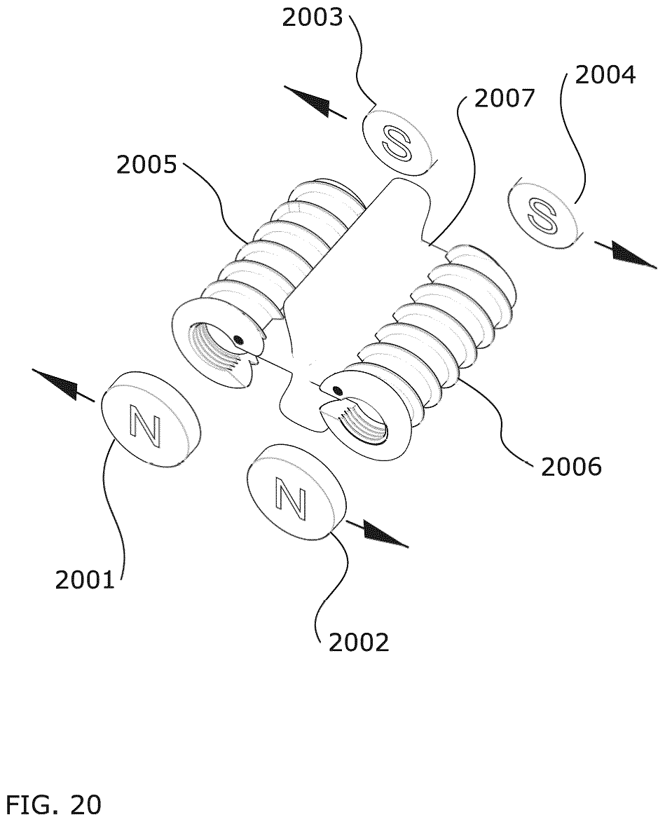

In certain embodiments as disclosed herein, the anchor is smooth rod with rivet cap (cap is locked into bone). (See FIG. 32). In certain embodiments as disclosed herein, the anchor is an allograft bone scaffold. In certain embodiments as disclosed herein, the anchor is a cannulated bone screw. (See FIG. 9). In certain embodiments as disclosed herein, the anchor has magnetic stenting via charged heads at both end magnetic end caps. (See FIG. 20). In certain embodiments as disclosed herein, the anchor has a slot in situ, and/or a premanufactured slot.

### Connector

As set forth above, in certain embodiments the stemless implantable device of the present disclosure comprises a flexible connector that spans the joint space between, for example, the proximal phalanx and the middle phalanx in the coronal plane and flexes in the sagittal plane, to serve as a joint replacement system. In certain embodiments, the connector, may be, for example, a flexible connector, in its various embodiments may be manufactured using, for example, a variety of fatigue-resistant biocompatible materials as disclosed herein, such as silicone, ultra-high molecular weight polyethylene (UHMWPE), nitinol shape memory alloy, titanium and carbon nanofibers, strips or plates of various thickness and width, and braided nanofiber elements woven in two dimensional and three dimensional arrays engineered to confer strength and flexibility as specified to meet the needs of the particular patient involved. The flexible connector may be designed to incorporate various ranges of strength, flexibility, elasticity and other material properties so as to optimize the path of motion of the phalanges and durability of the device.

In certain embodiments of the flexible connector, a preflexed connector has a differential bumper size and ratio to the silicone. In certain embodiments of the flexible connector, for example, silicone is custom printed in a mesh-like pattern to respond to stresses in unique ways.

In certain embodiments of the flexible connector, the in-Situ malleability may be adjusted post-implantation. In certain embodiments of the flexible connector there may be inherent adjustment capabilities to rectify misalignment caused by non-parallel relationship of proximal and distal anchors.

In certain embodiments of the flexible connector, there may be malleable material between anchor and flexible connector.

In certain embodiments of the flexible connector, due to variable density, curing, material properties, construction, geometry, manufacturing processes, and/or other factors, malleability comes from the flexible connector.

In certain embodiments of the flexible connector, the flexible connector may be comprised of a beam with no bumper; may impart stenting behavior to keep bone surfaces apart from each other, may have a memory to return to a pre-flexed neutral angle of flexion; and/or may have a graduated or variable density with variable characteristics of strength and flexibility.

In certain embodiments of the flexible connector, a kit comes with standard various angled implants to correct anchor misalignment. In certain embodiments of the flexible connector, a kit comes with various pre-flexed neutral angles of flexion.

In certain embodiments as disclosed herein, material characteristics of the flexible connector allow for smooth flexion within a normal range of zero to one hundred and ten (0-110) degrees when activated by the patient's flexor tendon system.

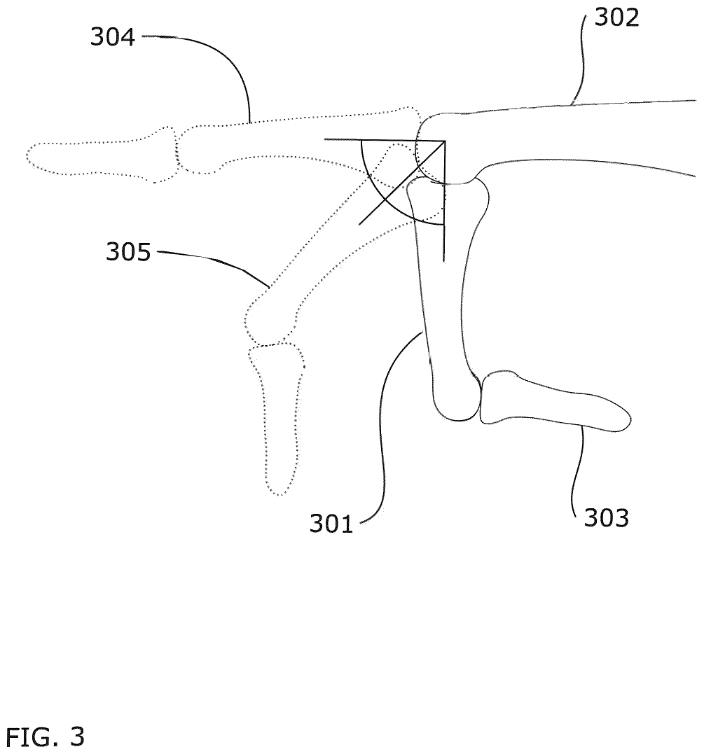

In certain embodiments as disclosed herein, a flexible planar component manufactured using one or more of a variety of fatigue resistant biocompatible materials configured to allow motion of the joint in the sagittal plane when activated by the patient's flexor tendon system and to allow constrained accessory motion when subject to external forces such as when used to assist in gripping an oddly shaped object. In certain embodiments as disclosed herein, the width is not to exceed the intracortical dimension measured in the coronal plane of the patient's bone at the site of implantation. In certain embodiments as disclosed herein, the path of motion in the sagittal plan may, for example, follow an arc path described by a normal interphalangeal joint or approximation thereof. (See FIG. 3). In certain embodiments as disclosed herein, motion in the coronal plane restricted to between about 0 and about 2 deg allowable motion. In a preferred embodiment, motion in the coronal plane restricted to about 0 deg allowable motion.

In some examples, the flexible connector can be a mesh hinge. In one embodiment, the flexible connector may be comprised of woven or laminated components to create a flexible mesh with a variety of dynamic qualities and fixation methods. Said “mesh connector” may be secured to the bones of the first and second phalange using a fixation system comprised of, for example, a rod pocket, or sleeve, that is integrally woven into the proximal edge and distal edge of said mesh connector.

In certain embodiments of the flexible connector, stiffness may be specified to meet the needs of the particular patient involved. For example, in the absence of a healthy extensor tendon system, a surgeon may specify a stiff “spring mesh” that passively returns to a straight position upon release by the patient's flexor tendon system, with no participation required by the extensor tendons. In this embodiment, mesh connector stiffness maintains proper joint spacing to prevent bone-on-bone contact of the articular surfaces of the first and second bones of the joint. As set forth above, bone-on-bone contact is a common cause of severe pain in the advanced stages of all forms of arthritis. Bone-on-bone contact leads to inefficient joint mechanics that impairs digital range of motion, accelerates the degenerative process, and may ultimately lead to an ankylosis or complete loss of motion at the joint.

Furthermore, in certain embodiments of the flexible connector, to prevent harmful bone-on-bone contact, said flexible connector may incorporate a transverse bumper to protect terminal surfaces of opposing bones of the joint from contact with the opposing bone of the joint.

The stiffer “spring mesh” embodiment may be achieved using a plain weave rather than a bias weave, and using a wide cross-sectional profile for warp components, which run perpendicular to the fixation pins or rods within the implant device. The spring mesh configuration confers lateral stability and alignment in the coronal plane without reliance on joint ligaments.

In the case of a complete and healthy tendon system and intact cartilage on the articular surfaces of the condyle heads, a surgeon may specify a more elastic mesh connector that stretches in flexion to replicate the natural glide path of a healthy joint, and contracts when the digit is actively extended by the patient's extensor tendon system. A “stretch mesh” or “flexible mesh” embodiment may be achieved, for example, by using weave patterns that change in length as tension is applied, such as a bias weave of wide flat sections (similar to weaves typically found in “finger traps”). Additionally, fiber diameter or cross-sectional profile may be specified to deliver a Young's modulus of elasticity suitable to the weave pattern so as to allow stretch capacity in flexion of approximately 25% of the unflexed mesh length. Alternatively, certain behaviors of the stretch mesh may be achieved through use of engineered polymers, as described below.

In a different embodiment, the flexible connector may be comprised of a polymer, such as silicone, so as to allow translation and stretch that approximate natural physiologic motion of the interphalangeal joint. In certain embodiments, silicone custom printed in engineered pattern or patterns to respond to stresses in unique ways, or in graduated density such as highest density within or near to the fixation system and lower density where greater flexibility is desired to achieve the desired trajectory path of motion. Said polymer connector may incorporate bumper flanges that are oriented in a perpendicular aspect to the coronal plane of the polymer connector to prevent bone-on-bone contact. Said bumper flanges may be gel-filled to allow for dynamic response under various tension and compression conditions.

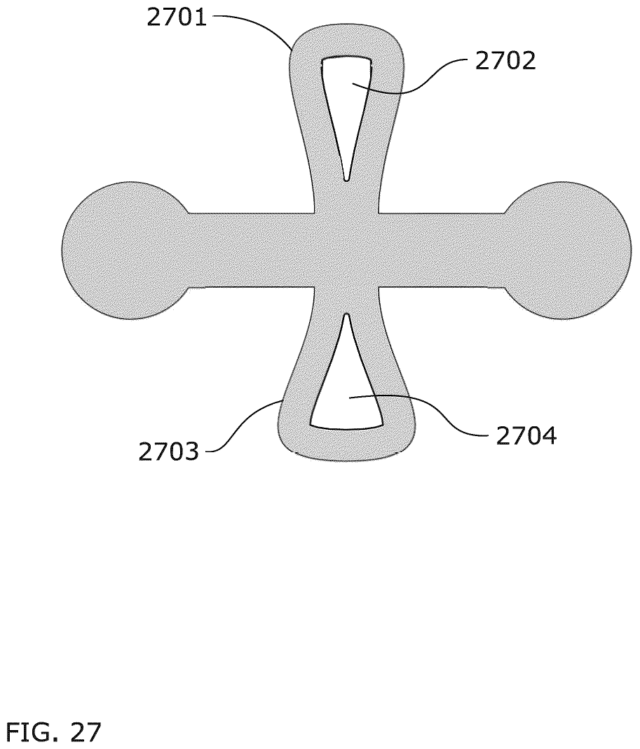

In certain embodiments, a bumper flange may incorporate hollow portion or gel-filled portion to allow translation of the second bone relative to the first bone along the arc of motion trajectory. (See FIG. 27 showing a cross sectional view of one embodiment of the flexible connector with integral transverse bumpers showing hollow a continuous opening within the profile of the dorsal and volar flanges of the bumper). In certain embodiments, the proximal and distal edge geometry configured as required for anchoring to mechanical anchor or directly within bone as described herein.

In the embodiments of the flexible connector described herein, including the mesh connector and the polymer connector which represent some of many possible materials and composite configurations that may be employed within the spirit and scope of the invention, flexion through the entire area of the flexible connector distributes bending stresses uniformly so as to minimize material fatigue. A plurality of weave patterns, laminations, or polymer curing formulae may be employed within a unitary flexible connector to allow differential stiffness, flexibility, strength, and fatigue resistance designed to manage variable stress patterns through various sections of the connector.

In certain embodiments as disclosed herein, the implant is stiff enough to, for example, resist forces in the coronal plane to, for example, optimize thumb to finger pinch. In certain embodiments as disclosed herein, though there may be some motion in the coronal plane, it will come from, for example, the material properties of the flexible connector.

In certain embodiments as disclosed herein, implant sequestration in the form of a pseudosynovial membrane may form around the flexible connector.

### Anchor—Connector Fastening

In certain embodiments as disclosed herein, the proximal fixation rod and/or the distal fixation rod are independently barbed, for example, to connect to the anchor. In certain embodiments as disclosed herein, the proximal fixation rod and/or the distal fixation rod independently comprise an anti-pullout peg.

In certain embodiments as disclosed herein, the proximal fixation rod and/or the distal fixation rod are independently non-rotating version(s), for example with thinner flutes.

In certain embodiments as disclosed herein, the proximal fixation rod and/or the distal fixation rod independently comprise an expanding hydrogel core to add stability during curing.

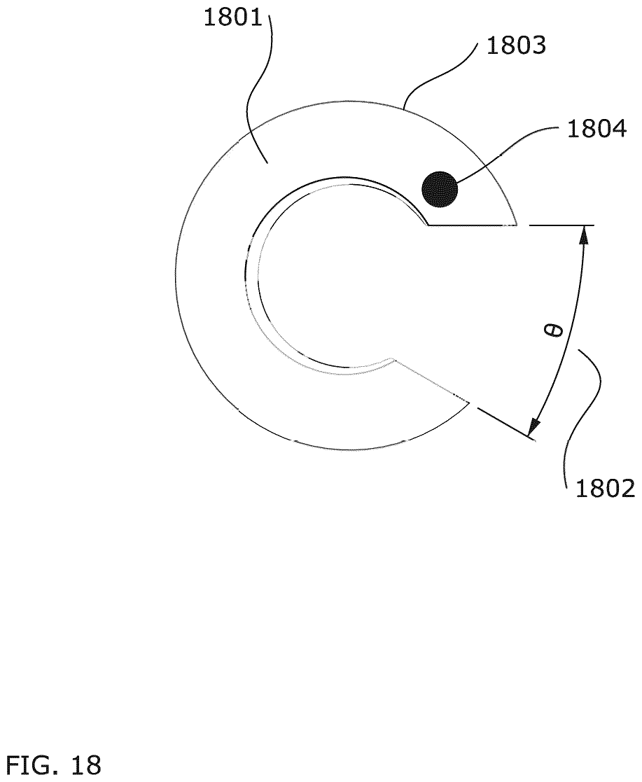

In certain embodiments as disclosed herein, the proximal fixation rod and/or the distal fixation rod are independently flute radians variants-optimized for rotation vs. pull out. For example, see the slot opening degree of radians labeled “theta” in FIG. 18. This radial dimension will vary depending on the flexible connector dimension and connector-anchor fixation type, the material properties of the flexible connector and the location of the anchor in the proximal position or distal position.

In certain embodiments as disclosed herein, the fluting of the anchor is, for example, a wedge-shaped notch in the anchor, that allows the flexible connector to exit the anchor and allows for some rotation of the connector during active motion. In certain embodiments as disclosed herein, the flute is designed to allow exit of the flexible connector and is designed as an angled “pie-slice” instead of a straight slot. This, for example, allows for rotation of the connector during finger flexion. This “pie-slice” cutout can be optimized so as to be big enough to allow for some rotation during flexion but will not be too big to allow easy disengagement of the connector from the anchor.

### Implant to Bone Interface

In certain embodiments as disclosed herein, the proximal fixation system and/or said distal fixation system independently comprise osteointegrated surface treatment. In certain embodiments as disclosed herein, the proximal fixation system and the distal fixation system independently comprises barbs configured and manufactured to break away under specific controlled mechanical action barbed press fit with breakaway barb. In certain embodiments as disclosed herein, the proximal fixation system and the distal fixation system independently mechanically fastened metal to cortical bone.

In certain embodiments as disclosed herein, the proximal fixation system and the distal fixation system independently use a reverse threading concept to increase pull out strength or simulate “lock-nut” style fastening or tightening.

In certain embodiments as disclosed herein, the proximal fixation system and the distal fixation system independently comprise an anchoring bone rivet or screw cap. (See FIG. 21). In certain embodiments as disclosed herein, the proximal fixation system and the distal fixation system independently comprise bone scaffold integration. In certain embodiments as disclosed herein, the proximal fixation system and the distal fixation system independently comprise magnetic construction.

In certain embodiments as disclosed herein, the proximal fixation system and the distal fixation system independently comprise electronic monitoring capabilities. In certain embodiments as disclosed herein, a proximal fixation system and/or a distal fixation system independently comprise biometric monitoring.

In certain embodiments as disclosed herein, the proximal fixation system and the distal fixation system are inserted directly into bone with no anchor, using a pin or hydrogel core. In certain embodiments as disclosed herein, the flexible connector may comprise bumpers, and the flexible connector may be inserted directly into bone.

### EXEMPLARY EMBODIMENTS (FIGURES)

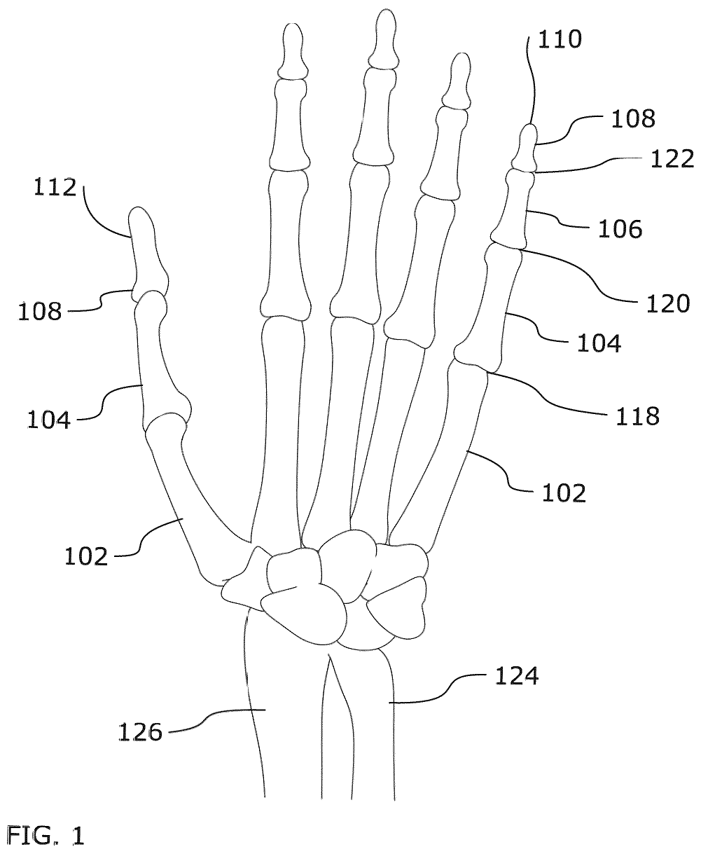

FIG. 1 shows an illustration of the bones of the hand, which is provided for reference to standard anatomical nomenclature referenced herein. The bones of the fingers comprise the metacarpal bones 102, proximal phalanges 104, middle (intermediate) phalanges 106, and distal phalanges 108, as numbered on the fourth finger (pinkie) 110. Bones of the thumb 112 include the metacarpal 102, proximal phalange 104, and distal phalange 108. There is no middle phalange in the thumb. The joint between the metacarpal bone and the proximal phalange is called the metacarpophalangeal joint, or MCP joint 118. The joint between the proximal phalange and middle phalange is called the proximal interphalangeal joint, PIP, or PIPJ 120. The joint between the middle phalange and the distal phalange is called the distal interphalangeal joint, or DIP 122. A biological joint typically includes a joint capsule 127 enclosing a joint space 128. The joint capsule includes dense tissues like connective tissue and collagen fibers, has a higher density than outside tissues (except bone and intra-articulate ligaments). The joint space is significantly less dense than the joint capsule.

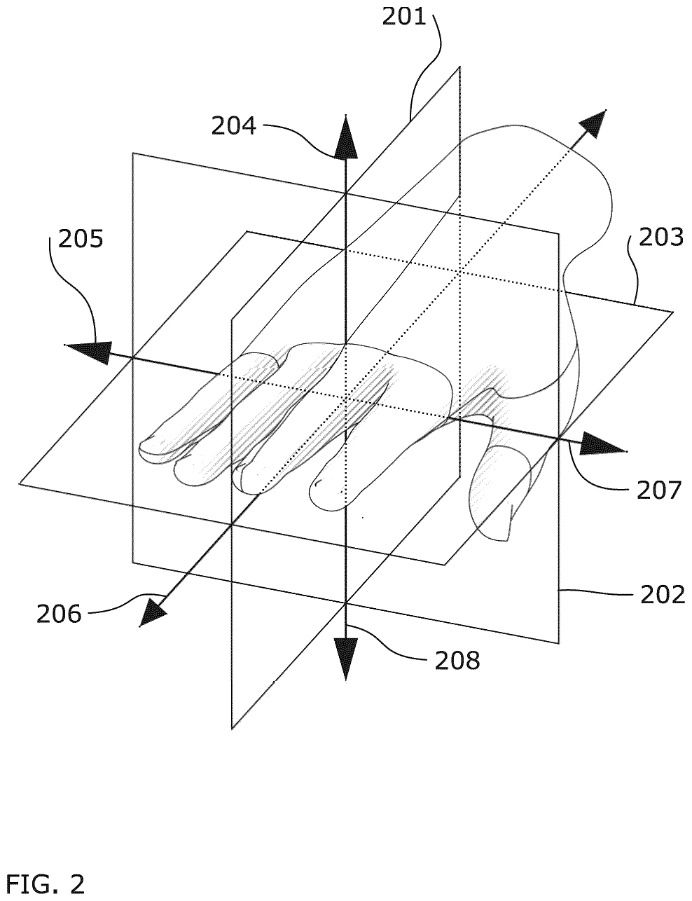

FIG. 2 shows the anatomical conventions of the directions through the hand. A plane from front to back through the centerline of the body is called the sagittal plane 201, which runs from front to back through the hand, from the dorsal side to the palmar side. A plane drawn across the hand is the transverse plane 202. A plane drawn through the side of the body or through the side of the hand perpendicular to the sagittal plane is called the coronal plane 203. The back of the hand is called the dorsal aspect 204. The palm side of the hand is called the palmar or volar aspect 208. The thumb-side of the hand adjacent to the radius bone of the forearm is called the radial aspect 207. The pinkie-side of the hand adjacent to the ulna bone of the forearm is called the ulnar aspect 205.

FIG. 3 depicts a perspective view of the anatomy at the proximal interphalangeal joint shown in a flexed position. The bones of the finger comprise the proximal phalanges 302, middle (intermediate) phalange 301, and distal phalange 303. The middle phalange is indicated in the partially flexed position 305 and the extended position 304.

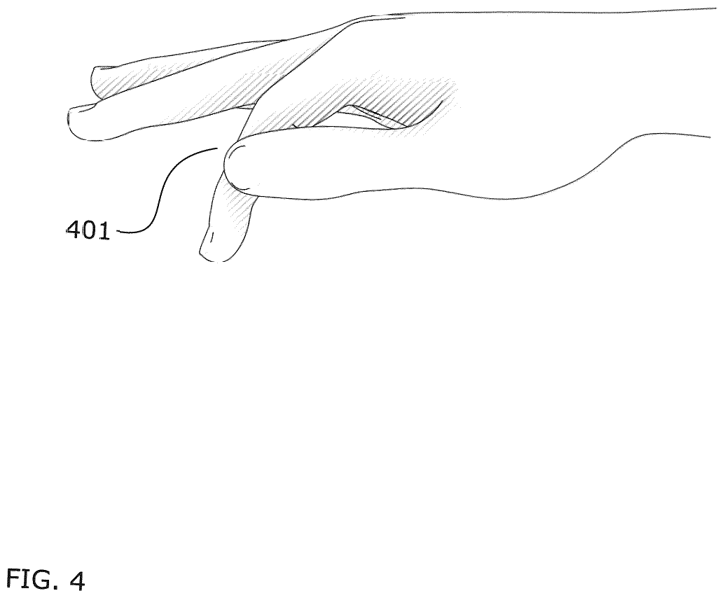

FIG. 4 depicts a perspective view of the hand with index finger in an extended position opposed by an axial force imposed by the thumb of the same hand indicating one type of accessory force resisted by the interphalangeal joint in ordinary use in a “key pinch” 401.

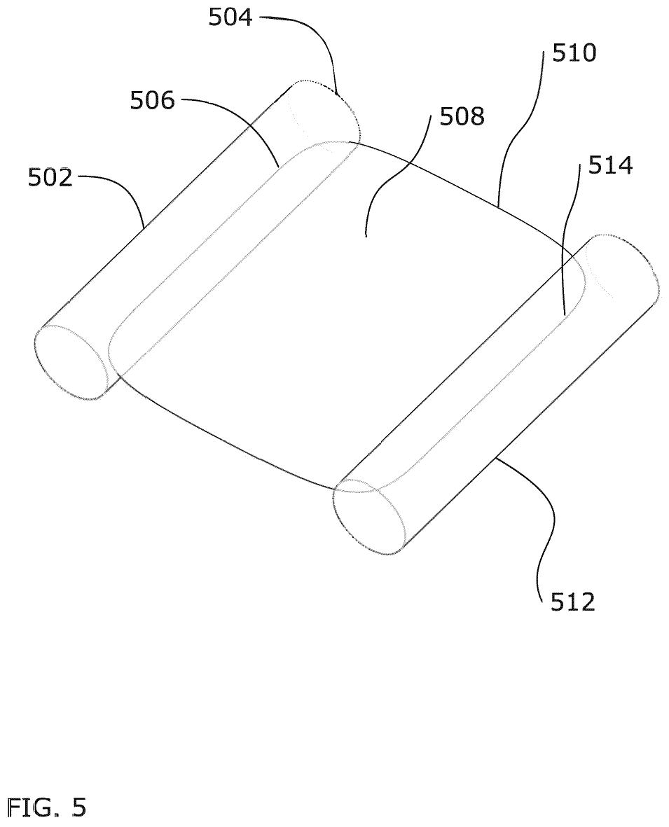

FIG. 5 depicts an illustration of a conceptual embodiment of the invention showing the proximal fixation rod or pin 502, the leading end 504 of said fixation rod or pin 502, the proximal edge 506 of a mesh hinge 508, the leading lateral edge 510, a distal fixation rod 512, and the distal edge 514 of the mesh hinge 508.

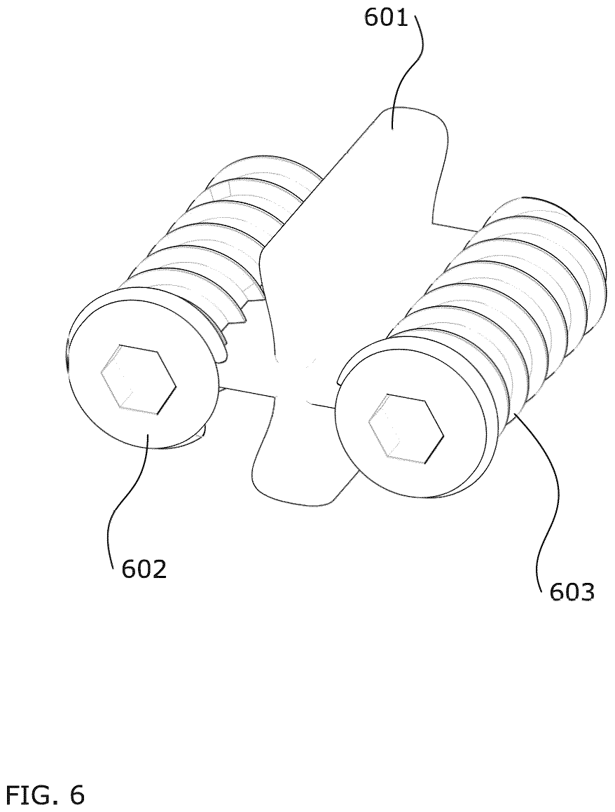

FIG. 6 depicts a perspective view of a proximal cannulated wide flange threaded anchor 603 with radial slot and reverse-threaded anchor cap 602 and a parallel distal connector of similar dimension and configuration aligned in a mirrored relationship about the medial axis of one embodiment of a flexible connector with dorsal and volar bumpers 601 and a continuous fixation boss at the proximal edge and distal edge of said flexible connector inserted into the proximal and distal anchors respectively and secured therein by one embodiment of an anchor cap screwed into the head of each anchor 602.

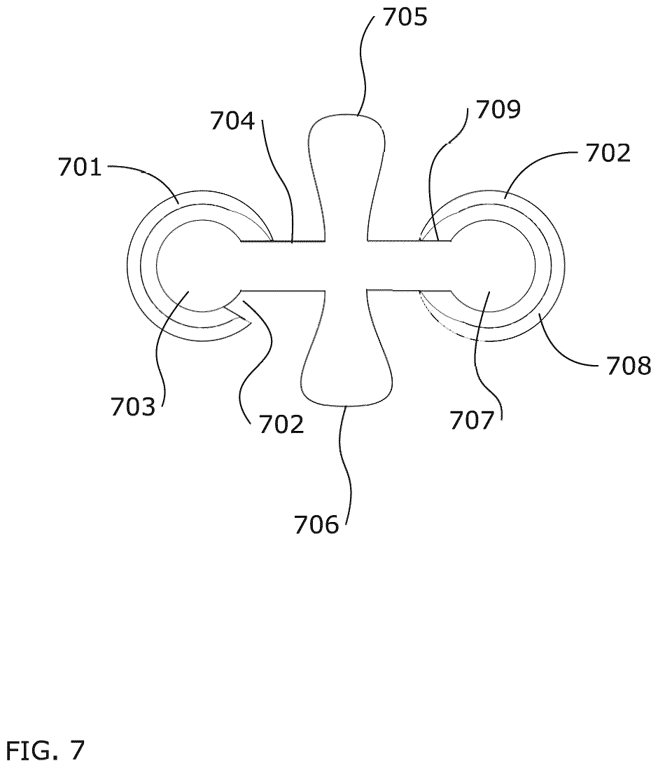

FIG. 7 depicts a cross sectional view of one embodiment of a flexible connector 704 in the extended (unflexed) position showing dorsal 705 and volar 706 bumper flanges and thickened barrels at proximal 703 and distal 707 edges and a keyhole slot 702 at the proximal condyle head. In this embodiment, the proximal edge 703 is shown inserted into the proximal fixation rod 701 and the distal 707 edge is shown inserted 709 into the distal fixation rod 702 which may have surface patterning 708.

FIG. 8 shows a top view of an exemplary embodiment of an implant device as disclosed herein showing a polymer connector 804 with ends 802 inserted into cannulated screw anchors 806. The head of the cannulated screw anchor 808, threads 807, cannula 805, cannula space 802 and end caps 808.

FIG. 9 shows an exploded perspective view of an implant device as disclosed herein showing cannulated screw anchors 902, 903, the head of the cannulated screw anchor 910, guide dot 911, a flexible connector 901 in the extended (unflexed) position, and threaded 913 end caps 904, 905.

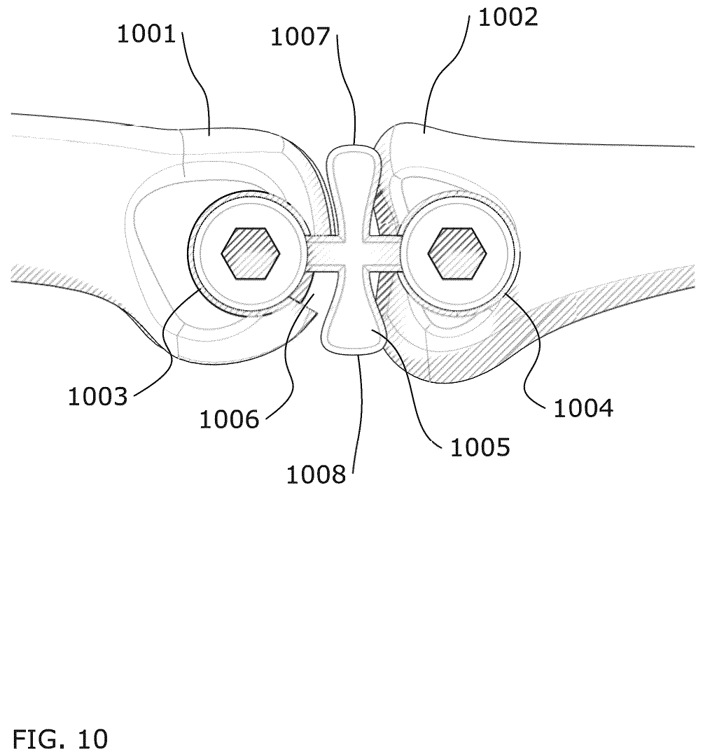

FIG. 10 depicts a cross sectional side view of the implant device as disclosed herein inserted into a proximal 1002 and distal 1001 phalange in the extended position, a polymer connector 1005 with dorsal 1007 and volar 1008 bumpers with ends inserted into cannulated screw anchors 1002, 1003. Also depicted is a keyhole slot 1006 at the distal condyle head.

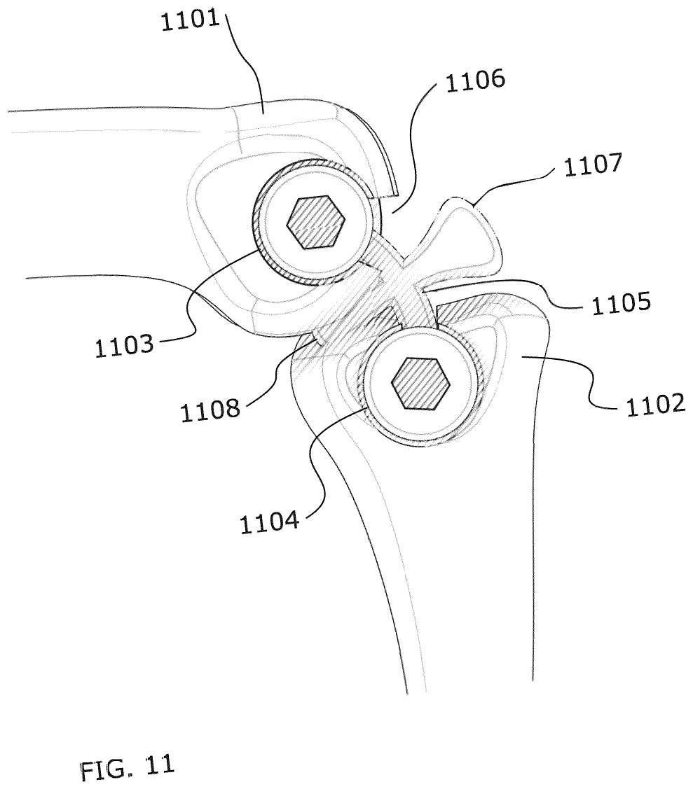

FIG. 11 depicts a perspective view of the implant device as disclosed herein inserted into a proximal 1101 and distal 1102 phalange in the flexed position, a polymer connector 1105 with dorsal 1107 and volar 1108 bumpers with ends inserted into cannulated screw anchors 1103, 1104. Also depicted is a keyhole slot 1106 at the proximal condyle head.

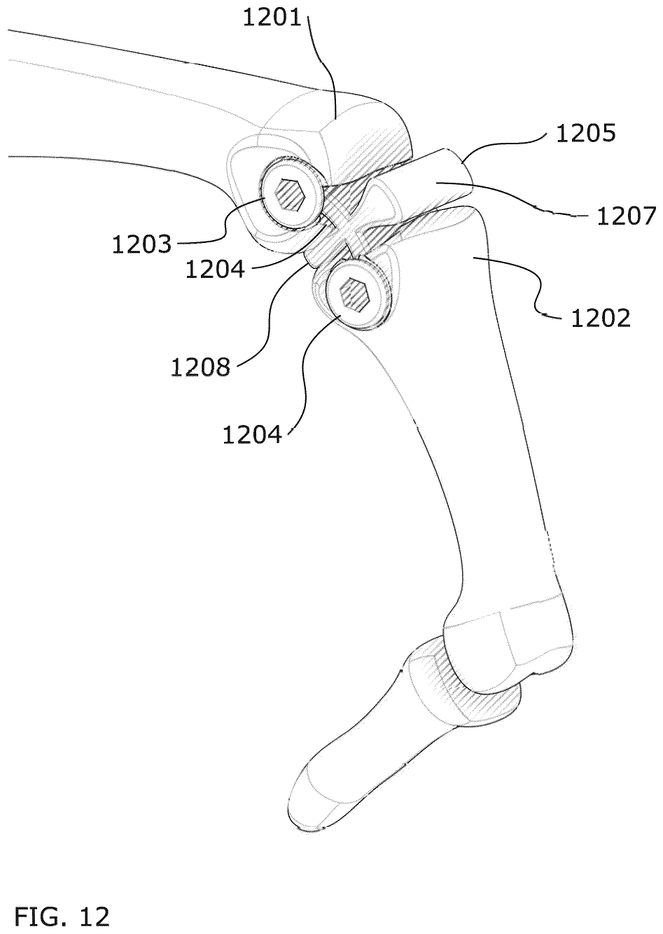

FIG. 12 depicts a perspective view of the implant device as disclosed herein inserted into a proximal 1201 and distal 1202 phalange in the flexed position, a polymer connector 1207 with dorsal 1205 and volar 1208 bumpers with ends inserted into cannulated screw anchors 1203, 1204. Also depicted is a keyhole slot 1204 at the proximal condyle head.

FIG. 13 depicts a cross sectional side view of the implant device as disclosed herein inserted into a proximal and distal phalange in the extended position, a polymer connector 1305 with dorsal 1303 and volar 1304 bumpers with ends directly inserted into bone 1301, 1302. Also depicted is a keyhole slot 1306 at the distal condyle head.

FIG. 14 depicts a top view of an exemplary embodiment of the implant device as disclosed herein inserted into a proximal 1403 and distal 1405 phalange in the extended position, a polymer connector 1404, and a magnetized component in the proximal transverse anchor 1401 in roughly parallel relationship with a magnetized component in the distal transverse anchor 1402 possessing the same magnetic polarity so as to exert a stenting force between the proximal and distal magnetized components.

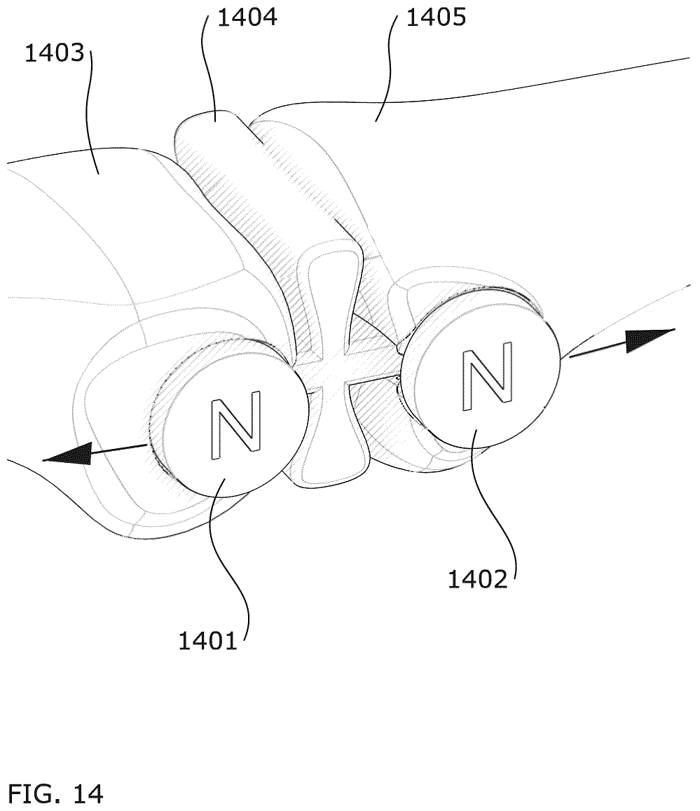

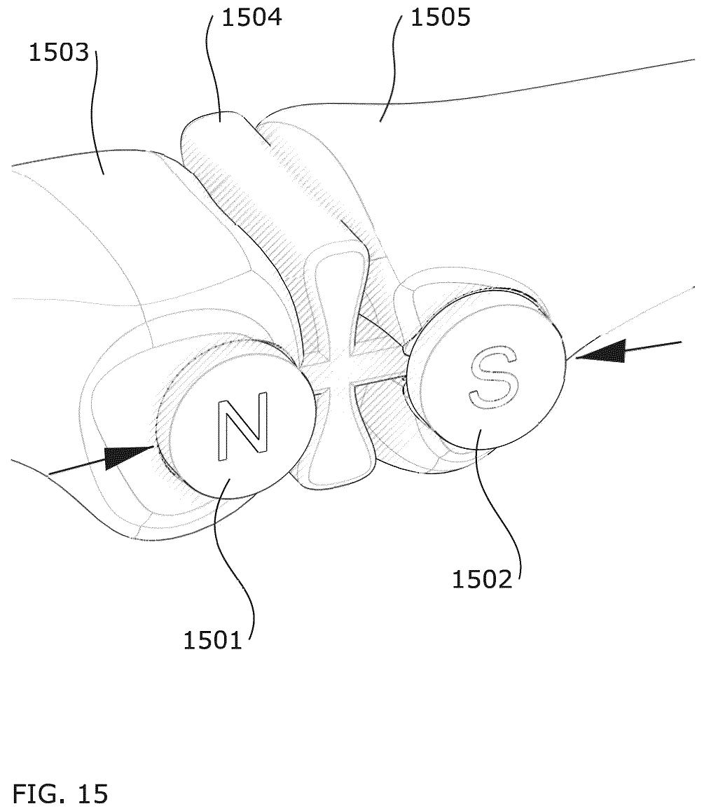

FIG. 15 depicts a top view of an exemplary embodiment of the implant device as disclosed herein inserted into a proximal 1503 and distal 1505 phalange in the extended position, a polymer connector 1504, and a magnetized component in the proximal transverse anchor 1501 in roughly parallel relationship with a magnetized component in the distal transverse anchor 1502 possessing an attractive magnetic polarity so as to exert a stabilizing force between the proximal and distal magnetized components.

FIG. 16 depicts a side view of one embodiment of a fixation rod 1601 showing a plurality of barbs 1602.

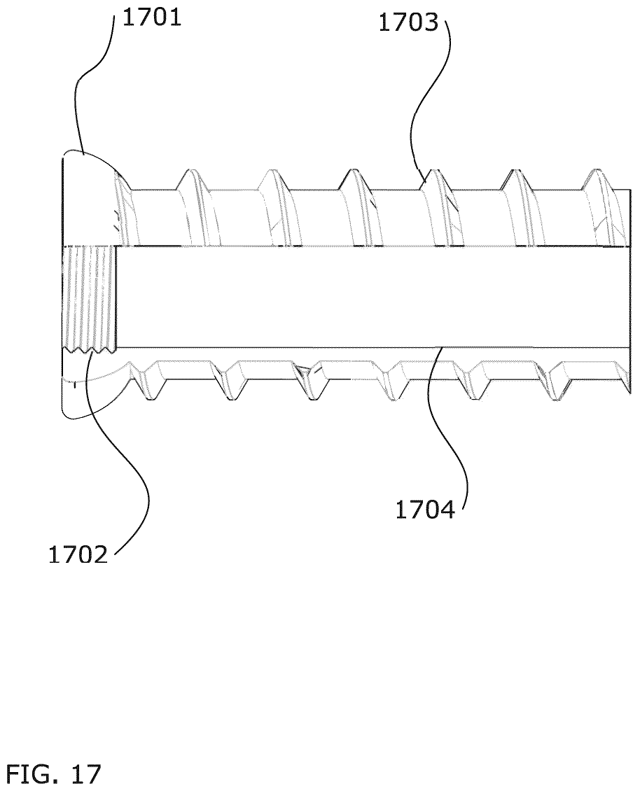

FIG. 17 depicts a side view of cannulated screw anchor, the head of the cannulated screw anchor 1701, threads 1703, cannula space 1704, and internal threads for attachment of end cap 1702.

FIG. 18 is a top cross section view of a cannulated screw anchor 1801, with threads 1803, guide dot 1804 and opening for flexible connector 1802.

FIG. 19 is a perspective view of a threaded 1901 end cap 1902.

FIG. 20 shows an exploded perspective view of an implant device as disclosed herein showing cannulated screw anchors 2005, threads 2006, a flexible connector 2007 in the extended (unflexed) position, and magnetic end caps 2001, 2002, 2003, 2004. In this exemplary embodiment, the transverse fixation system is inserted into a phalangeal bone using the surgical methods as set forth herein. In this exemplary embodiment, the transverse fixation system is made of a magnetic material and has a magnetic end cap.

FIG. 21 shows a perspective view of a flexible connector 2103 being inserted into a cannulated screw anchor 2102 with an end cap 2101.

FIG. 22 is a cross sectional view of an anchor 2202 with a guide dot 2204 and a flexible connector 2203 inserted into the anchor 2201.

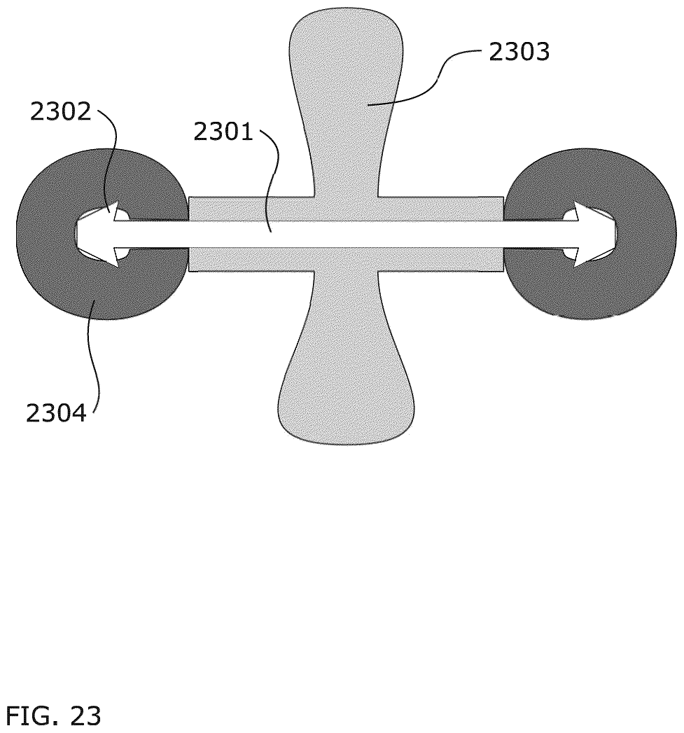

FIG. 23 is a cross sectional view of a flexible connector 2303 in the extended configuration illustrating an integrated rod pocket 2304, or sleeve, woven into the connector to receive the proximal rod and distal rod 2302 which are inserted into the respective proximal and distal rod sleeves.

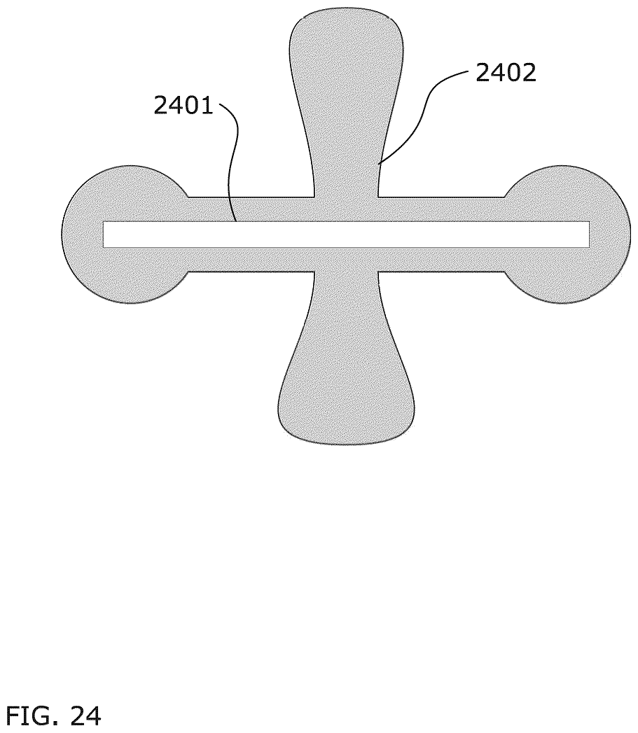

FIG. 24 is a cross sectional view of a flexible connector 2402 in the extended position with a solid insert 2401.

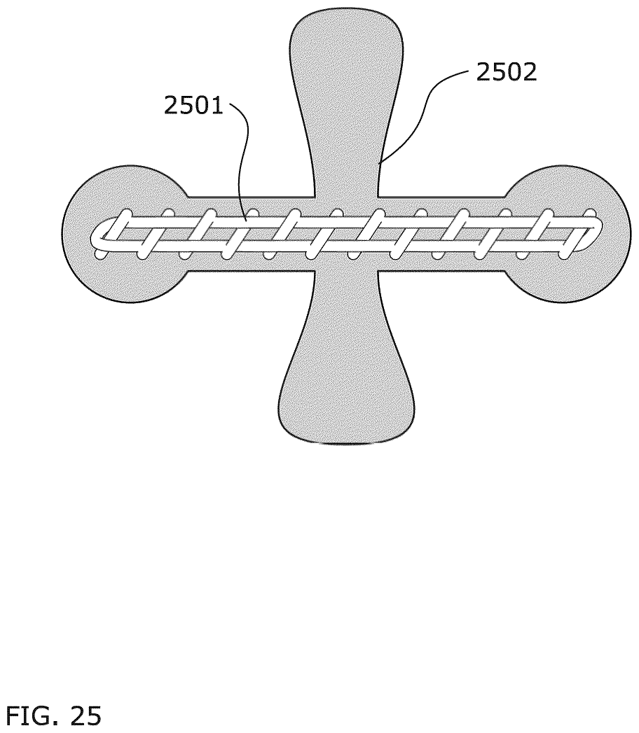

FIG. 25 is a cross sectional view of a flexible connector 2502 in the extended position with a mesh insert 2501.

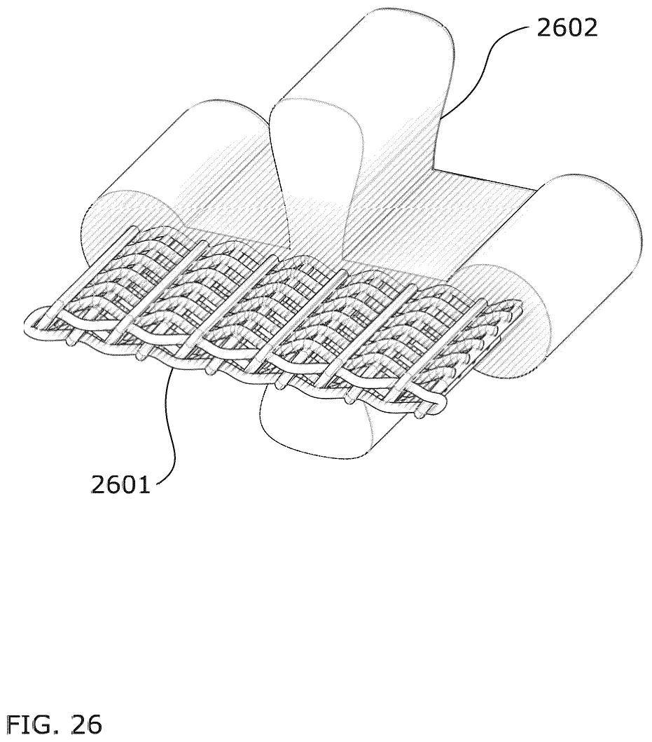

FIG. 26 is a perspective view of a flexible connector 2602 in the extended position with a mesh insert 2601.

FIG. 27 is a cross sectional view of a flexible connector with a hollow 2702 dorsal flange 2701 and a hollow 2704 volar flange 2703.

FIG. 28 is a perspective view of a surgical procedure for implantation of an implant device as disclosed herein, showing a guide wire 2802, external jig 2803, hollow drill bit 2804, hollowed out phalangeal bone 2805, and a transverse bone saw 2806.

FIG. 29 is a perspective view of guide wires 2901 being inserted into a phalangeal joint 2902.

FIG. 30 is a perspective view of a hollow drill bit 3002 using a guide wire 3001 to drill into phalangeal bone 3003.

FIG. 31 is a perspective view of an external jig 3101 guiding the insertion of guide wires 3102, 3103 into a phalangeal joint.

FIG. 32 is a perspective view of a smooth fixation rod 3201 with a rotatable end cap 3202, 3203 showing the direction of movement 3204; and a smooth fixation rod 3205 with a cuff-link style end cap 3206, 3207, showing the direction of movement 3208.

FIG. 33(A) is a perspective view of a fixation rod 3301 with fluting 3302. FIG. 33(B) is a perspective view of a fixation rod 3303 with surface treated to allow for osseointegration 3303.

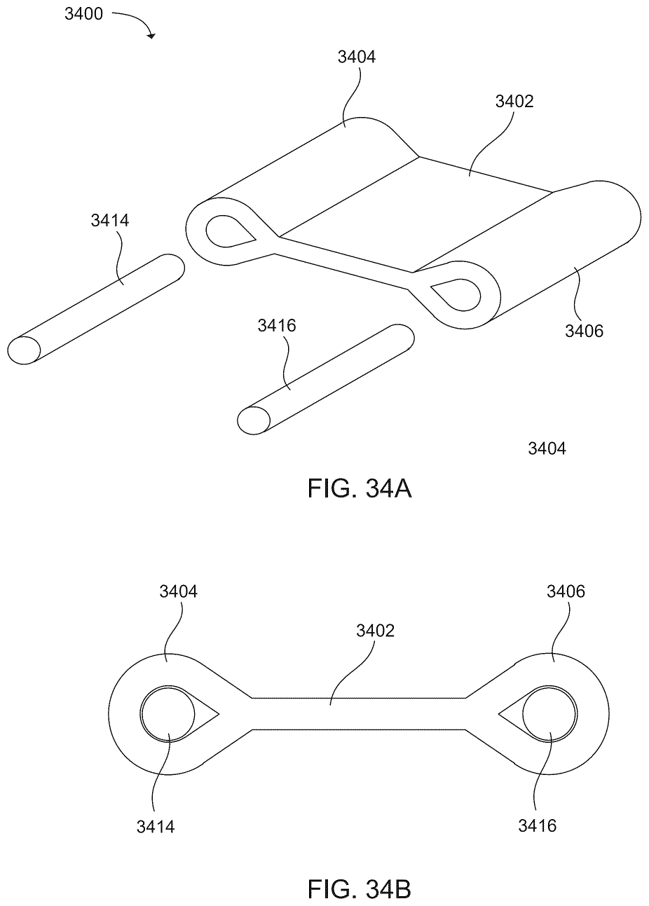

FIG. 34A is a perspective view of another example stemless implantable device for arthroplasty 3400. This device includes a flexible connector 3402 having a proximal rod sleeve 3404 integrated in a proximal portion of the flexible connector and a distal rod sleeve 3406 integrated in a distal portion of the flexible connector. A proximal fixation rod 3414 is configured to be inserted into an opening 3408 of the proximal rod sleeve. A distal fixation rod 3416 is configured to be inserted into an opening of the distal rod sleeve. FIG. 34B shows a side view of this example, after the proximal fixation rod has been inserted into the proximal rod sleeve and the distal fixation rod has been inserted into the distal rod sleeve. This flexible connector can be a type of flexible connector for use in any of the examples described above. In some examples, the flexible connector can be implanted directly into cavities formed in phalanges, such that the flexible connector contacts the bone. In other examples, the flexible connector can be implanted into any of the anchors described herein, such as cannulated screw anchors.

In some examples, the fixation rods can be inserted into the rod sleeves of the flexible connector before or after the flexible connector has been implanted. In some examples, the flexible connector can be implanted first without the fixation rods. Then, the fixation rods can be inserted into the rod sleeves to expand the rod sleeves. This can be useful to increases the thickness of the rod sleeves to facilitate retention of the rod sleeves in cavities formed in the phalanges, or in anchors that include a cannula and slot as described above. In certain examples, a bone cavity or an anchor can include a slot that is wide enough to allow the rod sleeves to pass through when the rod sleeves are empty, but not wide enough to allow the rod sleeves to pass through after the fixation rods have been inserted. In further examples, inserting the fixation rods can expand the rod sleeves sufficiently that the rod sleeves apply pressure to the bone cavities or anchors in which the rod sleeves are implanted. This pressure can increase friction to further facilitate retention of the rod sleeves in the bone cavities or anchors.

It is noted that the proximal rod sleeve and the proximal fixation rod are named after the proximal phalange. As the device is intended to be implanted at a join between two phalanges, the proximal phalange refers to the phalange that is nearer to the patient's body and the distal phalange refers to the phalange that is more distant from the patient's body. The proximal and distal phalanges can be any adjacent pair of phalanges present in any finger, thumb, or toe of the body. The proximal fixation rod and the proximal rod sleeve can be configured to be implanted in a proximal bore drilled in the proximal phalange. The distal fixation rod and the distal rod sleeve can be configured to be implanted in a distal bore drilled in the distal phalange. The flexible connector can be configured to connect the proximal fixation rod and the distal fixation rod within a channel excised through bone along a continuous path between the proximal bore and the distal bore. As used herein, “proximal bore” and “distal bore” are also named after the proximal phalange and distal phalange, respectively.

The proximal rod sleeve and distal rod sleeve can be open at both ends in some examples. The “ends” of the rod sleeves refers to the locations where the openings are present. When a fixation rod is inserted, the fixation rod can be inserted into an opening at one end of the rod sleeve and slide through the sleeve toward the opposite end of the rod sleeve. In other examples, the rod sleeves can be closed at one end and open at the other end, forming a pocket that the fixation rod can be inserted into. In such examples, the fixation rod can be inserted into the open end and pushed in until reaching the closed opposite end. In still further examples, the rod sleeves can be closed at both ends. In these examples, the fixation rods can be enclosed inside the rod sleeves at the time of manufacture, before the device is implanted. Various examples can also include various combinations, such as a proximal rod sleeve that is open at both ends and a distal rod sleeve that is closed at one end, or a proximal rod sleeve that is closed at one end and a distal rod sleeve that is closed at both ends, or any other combination of closed and open sleeves.

The fixation rods can be elongated, with a length that is greater than a diameter or width of the rods. The fixation rods used in this example can have any of the features and characteristics of fixation rods described above in other examples.

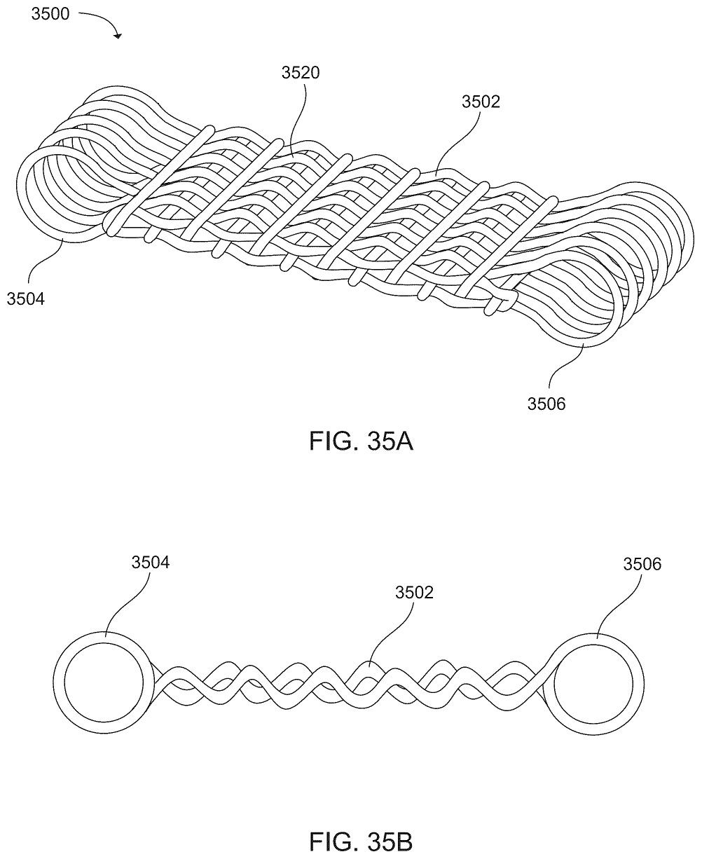

The flexible connector can be made out of any suitable mesh material. In some examples, the flexible connector can comprise a mesh hinge. Mesh materials can include interlaced components, such as fibers, threads, wires, plates, and so on. FIG. 35A shows a perspective view of another example flexible connector 3502 comprising a mesh pattern of wires 3520. This example also includes a proximal rod sleeve 3504 and a distal rod sleeve 3506. FIG. 35B shows a side view of this example. The side view shows that the proximal rod sleeve and the distal rod sleeve are both open at both ends to allow fixation rods to be inserted. Besides this example, a variety of other mesh patterns and materials can be used.

When the flexible connector is implanted in a joint between two phalanges, it can be useful to allow the flexible connector to stretch when the joint is flexed. Therefore, the mesh material can be stretchable. In some examples, the mesh can stretch up to a 25% increase in length, as mentioned above. The mesh can comprise fibers of a stretchable material in some cases, or in other cases non-stretching materials can be arranged in a mesh pattern that can be stretchable because of the pattern itself. In certain examples, the mesh can include materials such as medical grade plastic, bioactive materials, carbon nanofibers, carbon plates, carbon strips, composite materials, elastomeric polymers, hydroxyapatite (HA) coatings, super-elastic metal alloys, nitinol, internal shape memory alloy, polyetheretherketone (PEEK), silicone, titanium alloys, titanium, polyethylene (PE), engineered polymers, ultra-high molecular weight polyethylene, high molecular weight polyethylene, aromatic polyamide, polymers made to be radiopaque, materials derived therefrom, and combinations thereof. Any of the polymers could be made radiopaque by the addition of iodine or bromine-based monomers, or heavy-metal-containing monomers to obtain radiopaque polymer matrices so as to permit tracking of accumulated damage via x-ray, without having to have physical access to the implantable device.

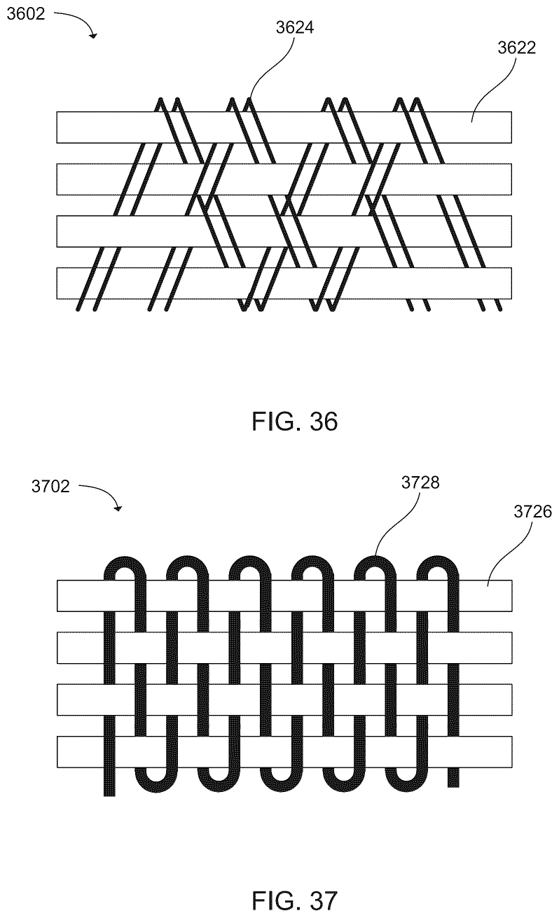

FIG. 36 shows a top-down view of an example flexible connector 3602 with a mesh pattern that includes longitudinal strips 3622 spaced across the width of the flexible connector. The longitudinal strips are woven together with a flexible material 3624. In some examples, the longitudinal strips can comprise titanium. Other metals, plastics, ceramics, or other materials can also be used for the longitudinal strips. In further examples, the flexible material can comprise carbon nanofibers. Other flexible fibers can also be used.

FIG. 37 shows a top-down view of another example flexible connector 3702. The mesh pattern is constructed using a plain weave that includes warp components 3726 and weft components 3728. The warp components can run perpendicular to the fixation rods (i.e., the warp components can extend along the length of the flexible connector) and the wet components can extend transversely across the width of the flexible connector. The warp components can be strips of material, such as titanium, that have a cross-sectional profile with a greater width than thickness. In other words, the wrap components can be thin, flat strips of material. This mesh can confer lateral stability and alignment in the coronal plane without reliance on joint ligaments.

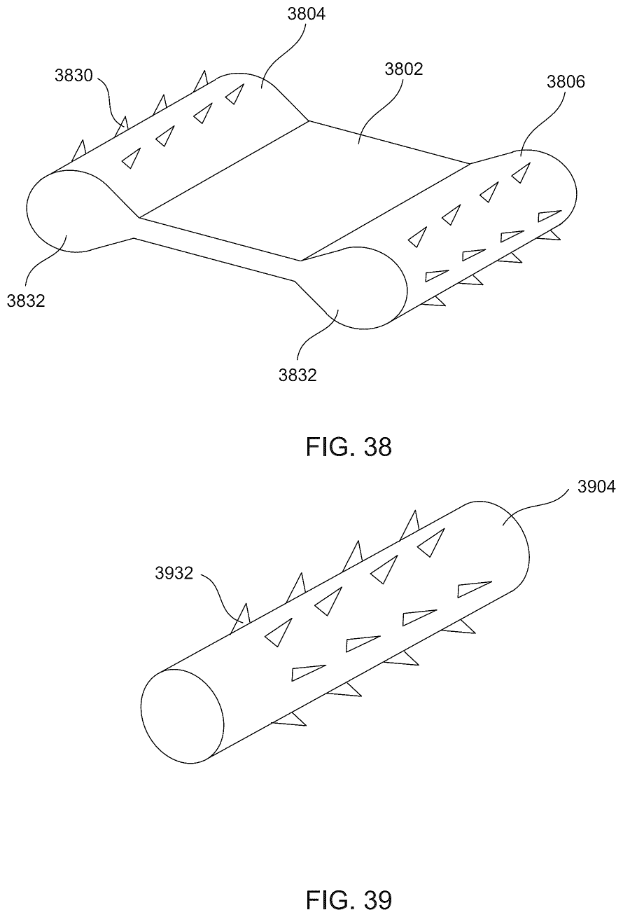

FIG. 38 is a perspective view of another example flexible connector 3802. This example comprises barbs 3830 on the exterior surfaces of the proximal rod sleeve 3804 and the distal rod sleeve 3806. The barbs can facilitate retention of the flexible connector by securely engaging with bone into which the flexible connector is implanted. If the flexible connector is implanted into anchors, in some examples the barbs can also be useful to securely engage with the interior surface of the anchors. The barbs can be slanted away from an insertion end of the rod sleeve so that the rod sleeve can be inserted easily, but the barbs can prevent the rod sleeve from being pulled back out in the opposite direction after being inserted. This example also includes closed ends 3832 on both the proximal and the distal rod sleeve. The opposite ends of the rod sleeves can be open to allow fixation rods to be inserted. Alternatively, the opposite ends of the rod sleeves can also be closed and the fixation rod can be enclosed within the rod sleeves before implantation.

The fixation rods can also include barbs, which can be useful to securely engage with the interior of the rod sleeves of the flexible connector. FIG. 39 shows an example fixation rod 3904 that includes barbs 3932. The barbs can be slanted away from the insertion end of the rod, so that the rod can be slid into the rod sleeve easily and the barbs can engage with the interior of the rod pocket to prevent the rod from being pulled back out in the opposite direction.

The fixation rods can be made of a suitable biocompatible material. Some examples of biocompatible materials can include medical grade plastic, bioactive materials, carbon nanofibers, carbon plates, carbon strips, composite materials, elastomeric polymers, hydroxyapatite (HA) coatings, super-elastic metal alloys, nitinol, internal shape memory alloy, polyetheretherketone (PEEK), silicone, titanium alloys, titanium, polyethylene (PE), ceramics, engineered polymers, ultra-high molecular weight polyethylene, high molecular weight polyethylene, aromatic polyamide, nickel-based superalloys, cobalt-chromium alloys, nitinol alloys, materials derived therefrom, and combinations thereof. In certain examples, the fixation rods can comprise a titanium material or a ceramic material. Furthermore, any polymers used to form the fixation rods could be made radiopaque by the addition of iodine- or bromine-based monomers, or heavy-metal-containing monomers to obtain radiopaque polymer matrices so as to permit tracking of accumulated damage via x-ray, without having to have physical access to the implantable device.

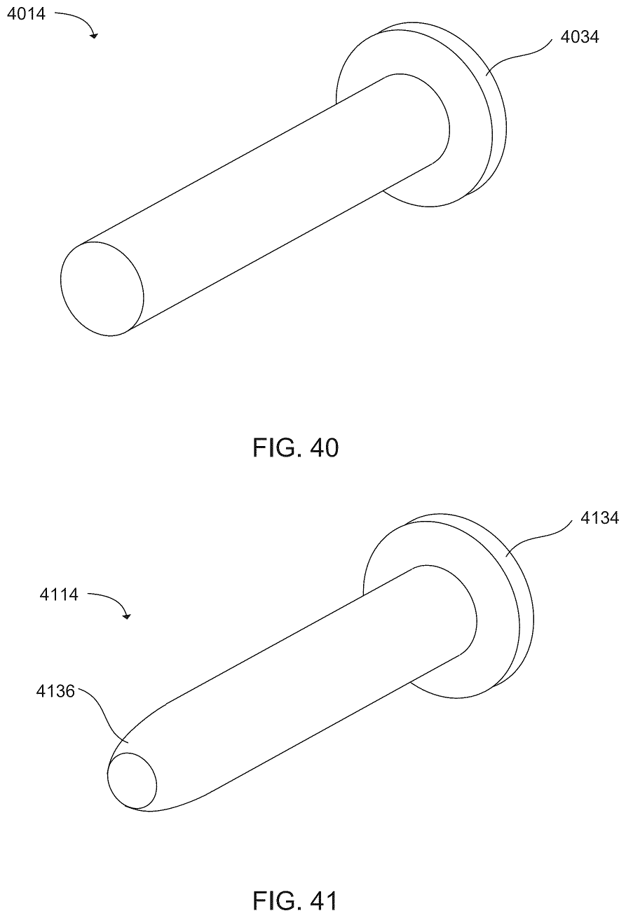

FIG. 40 shows a perspective view of an example fixation rod 4014 that has a smooth rod surface and an integrated end cap 4034. The integrated end cap can extend outward to a larger diameter than the diameter of the remainder of the fixation rod. The diameter of the end cap can be larger than the diameter of the rod sleeve in which the rod is to be inserted, to prevent the end cap from entering the rod sleeve. In some examples, the fixation rod can be inserted until the end cap abuts a bone surface around a bore drilled in a phalange, where the rod sleeve and fixation rod are implanted into the bore. In other examples, the rod sleeve and fixation rod can be implanted into an anchor that has previously been implanted into the bone, and the end cap can abut an end of the anchor.

FIG. 41 is a perspective view of another example fixation rod 4114 that also has an integrated end cap 4134. This fixation rod has a tapered end 4136 at the end which is to be inserted into the rod sleeve. The tapered end can facilitate insertion of the fixation rod into the rod sleeve. In some examples, the rod sleeve can also have a tapered end to facilitate press fitting of the rod sleeve into an implant site, such as a bore drilled in a phalange.

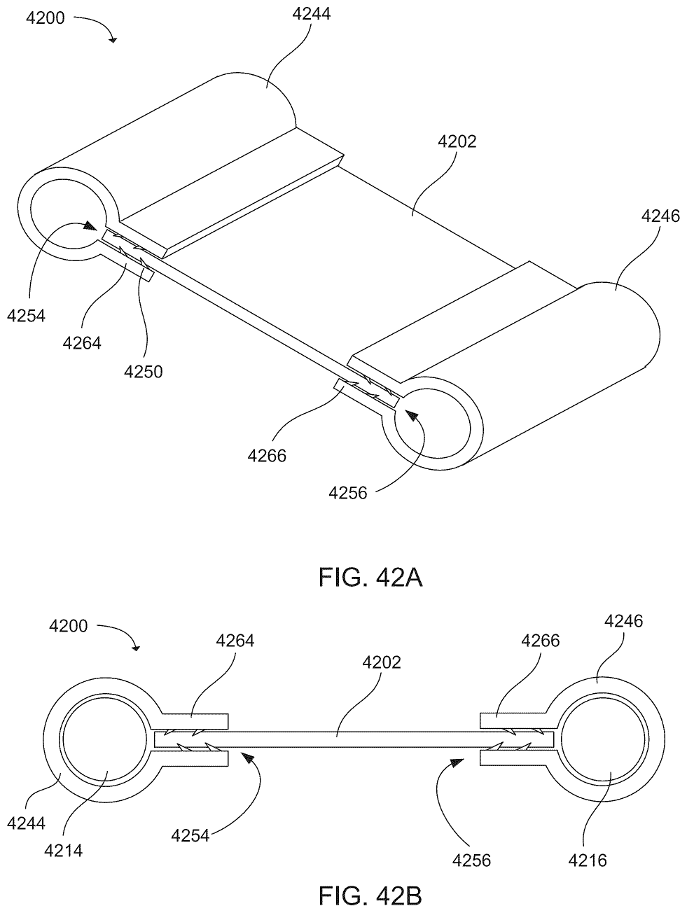

FIG. 42A is a perspective view of another example stemless implantable device for arthroplasty 4200. In this example, a mesh hinge 4202 is held between a proximal fixation hollow cylinder 4244 and a distal fixation hollow cylinder 4246. In this example, the mesh hinge does not include rod sleeves. Instead, the mesh hinge is a flat piece of mesh material. The mesh hinge can have any of the features and characteristics of mesh hinges described above. The proximal fixation hollow cylinder includes a proximal opening 4254 on a side of the proximal fixation hollow cylinder and proximal flanges 4264 framing the proximal opening. In this example, the one proximal flange extends outward above the proximal opening, and the other proximal flange extends outward below the proximal opening. The proximal flanges can be substantially parallel with a gap between the flanges sufficient to accommodate the mesh hinge. The flanges in this example include barbs 4250 on interior surfaces of the flanges (i.e., the surfaces in the gap facing toward one another). The barbs can securely engage the mesh hinge to prevent the mesh hinge from pulling out of the gap between the flanges. The distal fixation hollow cylinder also includes a distal opening 4256 and distal flanges 4266 framing the distal opening. The distal end of the mesh hinge can be held by the distal flanges.

It is noted that the terms “proximal fixation hollow cylinder” and “distal fixation hollow cylinder” are named after the proximal phalange and the distal phalange into which these fixation hollow cylinders are to be implanted. Similarly, the “proximal opening” refers to the opening of the proximal fixation hollow cylinder and “distal opening” refers to the opening of the distal fixation hollow cylinder. The “proximal flanges” are the flanges of the proximal fixation hollow cylinder and the “distal flanges” are the flanges of the distal fixation hollow cylinder. Thus, the terms “proximal” and “distal” are used to identify which fixation hollow cylinder these features belong to, and not some other difference between the openings or flanges. The entire device including the mesh hinge, proximal fixation hollow cylinder, and distal fixation cylinder, can be used in place of any of the flexible connectors in any of the examples described above.

FIG. 42B shows a side view of the example stemless implantable device for arthroplasty 4200 shown in FIG. 42A. The fixation hollow cylinders can be implanted into a cavity of a bone or into an anchor as described above. The fixation hollow cylinders can be rigid or semi-rigid so that they retain their shape and allow the device to be retained in the bone cavity or the cannula of an anchor. In some examples, the fixation hollow cylinders can be used without fixation rods inserted into the cylinders. However, in other examples, fixation rods can be inserted into the space within the fixation hollow cylinders. FIG. 42B shows a proximal fixation rod 4214 inserted into the proximal fixation hollow cylinder 4244 and a distal fixation rod 4216 inserted into the distal fixation hollow cylinder 4246. The fixation rods can have any of the characteristics and features described above.

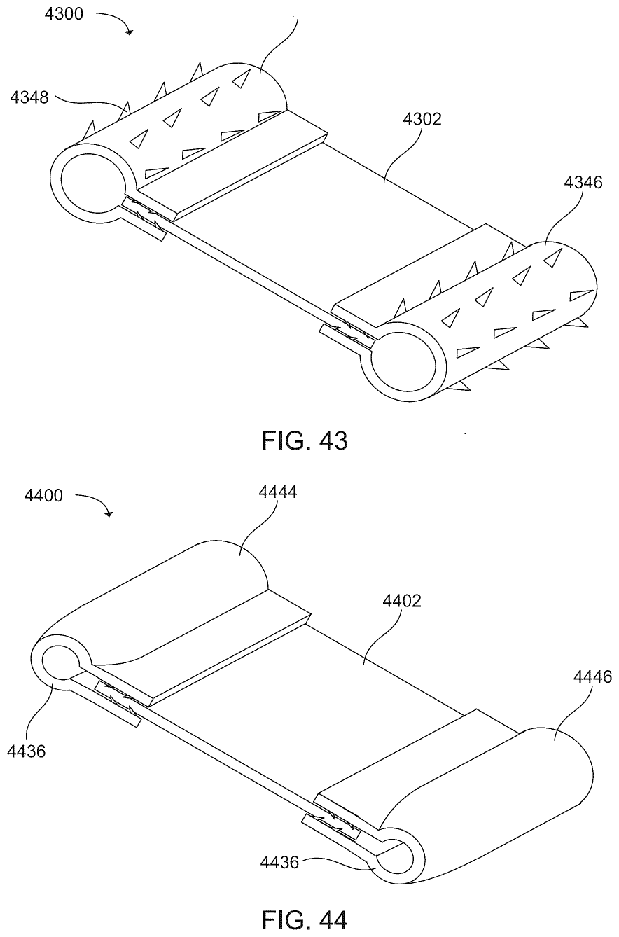

FIG. 43 is a perspective view of another example stemless implantable device for arthroplasty 4300. This example includes a mesh hinge 4302 held between a proximal fixation hollow cylinder 4344 and a distal fixation hollow cylinder 4346. The proximal fixation hollow cylinder and the distal fixation hollow cylinder include barbs 4348 on an exterior surface of the cylinders. These barbs can securely engage with bone when the cylinders are pressed into a cavity in a bone. The barbs can be configured in any way, for example, they can be configured to be slanted away from an insertion end of the cylinders, so that the cylinders can be pressed into the bone but the barbs can prevent the cylinders from pulling out of the bone. The barbs can further comprise different sizes and shapes.

FIG. 44 is a perspective view of another example stemless implantable device for arthroplasty 4400. This example also includes a mesh hinge 4402 held between a proximal fixation hollow cylinder 4444 and a distal fixation hollow cylinder 4446. In this example, the proximal fixation hollow cylinder and the distal fixation hollow cylinder have a tapered end configuration, with an insertion end being tapered to facilitate press fitting the proximal fixation hollow cylinder and the distal fixation hollow cylinder into bores drilled in the proximal phalange and the distal phalange, respectively.

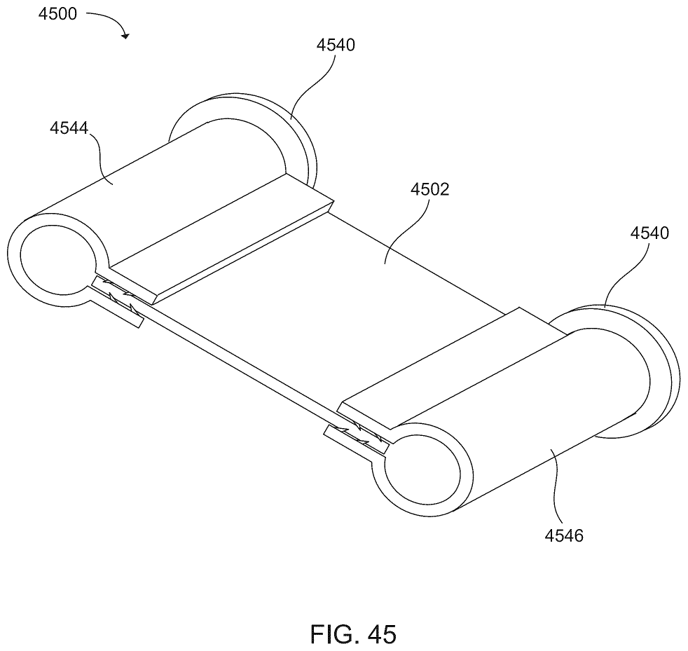

FIG. 45 is a perspective view of another example stemless implantable device for arthroplasty 4500. This example includes a mesh hinge 4502 held between a proximal fixation hollow cylinder 4544 and a distal fixation hollow cylinder 4546. The proximal fixation hollow cylinder and the distal fixation hollow cylinder in this example include integrated end caps 4540. The end caps extend outward from the cylinders to a diameter greater than the diameter of the remainder of the cylinders. In some examples, each fixation hollow cylinder can be pressed into a bore drilled in a phalange until the end cap abuts a bone surface around the bore. In other examples, the fixation hollow cylinder to be inserted into an anchor, and the end cap can abut an end of the anchor.

### Surgical Methods

The disclosure provides for surgical methods wherein, for example, surgical access and preparation of the implantation site is achieved through a single mid-lateral incision without disturbance to tendon systems. The transverse implantation method as disclosed herein allows for using minimally invasive surgical access via a mid-lateral incision. The mid-lateral approach avoids the need to interfere with flexor and extensor tendon systems during surgery and allows the joint to be exercised immediately upon removal of sutures without stressing the surgical wound.

Embodiments of the present disclosure permit an approach to treatment that is surgically simple, does not risk failure from bone loosening, and is not contraindicated in patients suffering from osteoarthritis, inflammatory arthritis (e.g., Rheumatoid arthritis, psoriatic arthritis, lupus, gout, pseudogout), or traumatic arthritis. Beneficially, embodiments of the present invention do not depend on ligaments for stability and do not rely on extensor tendons for proper function. Therefore, embodiments of the present disclosure may be used to reverse a small joint fusion, and feasibly may be revised without significant risk of bone failure leading down the path toward amputation.

Said incision is no longer than necessary to expose the lateral face of the joint capsule and to release angular deformities and flexion contractures. Following proper alignment, the bones are prepared to receive the implant device by drilling parallel holes and then sawing a continuous straight slot between said holes. The device is press-fit into the prepared bone openings and then the wound is closed. Motion exercises under the care of a therapist begin at the first post-operative appointment once sutures are removed.

The procedure is done under sterile conditions in an operating room. Preparation begins with decontamination of the upper extremity with surgical prep above the elbow. An upper extremity tourniquet or finger tourniquet may be used to facilitate hemostasis during the procedure.

The patient is draped in a sterile fashion leaving the operative extremity exposed (use of an extremity drape is encouraged.) The arm is elevated, exsanguinated and the tourniquet is inflated to a pressure of 250 mm Hg, or the finger tourniquet is applied. A mid-lateral incision is made over either the radial or ulnar aspect of the finger joint depending on the surgeon's preference. A drill is used to create parallel holes spanning the joint and without penetrating cortical bone on the opposite side. A sagittal saw is used to create a thin channel that connects the aforementioned parallel drilled holes. If indicated, a burr is used to resect a portion of condyle heads that show evidence of damaged cartilage or articular surfaces. Resected bone material, if any, is retained for possible use in capping anchor holes after insertion of the implant.

The implant is sized for width, using a depth gauge, selected, and press fit into the prepared bone spaces. The implant can include any of the stemless implantable devices for arthroplasty described herein. In some examples, the implant can include a device with a flexible connector having a proximal rod sleeve integrated in a proximal portion of the flexible connector and a distal rod sleeve integrated in a distal portion of the flexible connector. In other examples, the implant can include a mesh hinge that is held by a proximal flange of a proximal fixation hollow cylinder at a proximal end of the mesh hinge, and held by a distal flange of a distal fixation hollow cylinder at a distal end of the mesh hinge.

In another embodiment of the procedure using a cannulated screw anchor, the anchor is sized and countersunk below the surface of the cortical bone. After insertion of the implant into the proximal and distal cannulated screw anchors, said countersunk anchors are capped with resected bone flush with adjacent cortical surface. The wound is irrigated, and skin is closed with a monofilament suture. Motion exercises under the care of a therapist begin at the first post-operative appointment once sutures are removed.

FIG. 28 depicts a flow diagram of the detail components of the access and implant phases of the surgical procedure.

Skin and soft tissue preparation comprises the following steps. First, a #15 scalpel blade is used to incise the skin 2801. Blunt dissection is carried down to the transverse retinacular ligament which is divided. The lateral band can be divided as well if more exposure is required. Tendons are undisturbed. Angular or contracture deformities can be corrected at this point by releasing taught soft tissues including scarred or contracted collateral ligaments and/or the volar plate. Adequate soft tissue release is confirmed by demonstrating full passive flexion and extension in the sagittal plane.

Bone is prepared by resecting a portion of condyle heads that show evidence of damaged cartilage or articular surface. Resected bone material, if any, is retained for use in capping anchor holes after insertion of the implant.

The width of the distal bone will determine the width of the implant selection and will set the depth of the rod holes and mesh channel. To begin the drilling procedure 2802, a marked guidewire is drilled transversely parallel to the head of the proximal bone (e.g., proximal phalange) and in line with the approximate axis of rotation. The far cortex should not be perforated. Intraoperative fluoroscopy may be used to confirm the appropriate pin trajectory. A protruding pin is inserted into the guide hole. Then, a parallel drill guide set to a distance equal to the length of the implant device is placed over the first pin and used to drill a second pin transversely through the distal bone at a precise distance away and parallel to said first pin. In this step, care should be taken to avoid perforating the far cortex.

Next, the parallel guide is removed. The shorter of the two guide wire measurements is selected as this will determine the depth of drilling (e.g., 15 mm.) A cannulated three millimeter (3 mm) marked drill bit may be used to drill over each guide wire to the predetermined depth. A sagittal saw with blade thickness of one millimeter (1 mm) is then used to excise a continuous straight channel between the center points of the two bores 2806. Again, care should be taken to avoid perforating the far cortex.

Additional bone preparation involves resecting a keyhole shaped opening that expands the sagittal channel toward the volar aspect of the first phalange only.

In certain examples, a stemless implantable device for arthroplasty including a flexible connector can be press-fit into the bores formed in the phalanges. The flexible connector can include a proximal rod sleeve that is press-fit into the proximal phalange, and a distal rod sleeve that is press-fit into the distal phalange. The central portion of the flexible connector can extend through the channel formed in the bones between the two bores. In further examples, the rod sleeves can be press-fit into the bores and then fixation rods can be inserted into the rod sleeves after the rod sleeves are in the bores. In other examples, the stemless implantable device can include a mesh hinge, a proximal fixation hollow cylinder, and a distal fixation hollow cylinder. The proximal fixation hollow cylinder can be press-fit into the bore formed in the proximal phalange, and the distal fixation hollow cylinder can be press-fit into the bore formed in the distal phalange. The proximal and distal fixation hollow cylinders can include flanges to hold the mesh hinge between the cylinders, as described above. In some examples, the mesh hinge can be inserted into the flanges before the device is implanted. In other examples, the proximal and distal fixation hollow cylinders can be press-fit into the bores formed in the phalanges, and the mesh hinge can then be inserted into the flanges afterward.

In another embodiment of the procedure using a cannulated screw anchor, the anchor is sized and countersunk below the surface of the cortical bone.

An implant device of proper size is selected based on the depth of drilling or anchor device. The selected implant device may be press fitted into the prepared bone cavities or anchor device.

If cannulated screw anchors are used in lieu of the fixation rod or pin, after insertion of the implant into the proximal and distal cannulated screw anchors, said countersunk anchors are preferably capped with the patient's own resected bone, and said resected bone caps are then trimmed flush with the adjacent cortical surface.

The wound is then irrigated, and the skin is closed with a monofilament suture.

Preferably, motion exercises under the care of a therapist begins at the first post-operative appointment after the sutures are removed.

The invention will be illustrated in more detail with reference to the following Examples, but it should be understood that the present invention is not deemed to be limited thereto.

### Methods of Configuring an Implantable Device for Arthroplasty

In another example, a method of configuring an implantable device for arthroplasty can include providing a flexible connector having a proximal rod sleeve integrated in a proximal portion of the flexible connector and a distal rod sleeve integrated in a distal portion of the flexible connector, providing a proximal fixation rod, and providing a distal fixation rod. The method can further include configuring the proximal fixation rod to be inserted into the proximal rod sleeve, and configuring the distal fixation rod to be inserted into the distal rod sleeve. The method can also include configuring the proximal fixation rod and the proximal rod sleeve to be implanted in a proximal bore drilled in a proximal phalange. Similarly, the method can also include configuring the distal fixation rod and the distal rod sleeve to be implanted in a distal bore drilled in a distal phalange. The method can also include configuring the flexible connector to connect the proximal fixation rod and the distal fixation rod through a channel excised through bone along a continuous path between the proximal bore and the distal bore.

In another example, a method of configuring an implantable device for arthroplasty can include providing a proximal fixation hollow cylinder, providing a distal fixation hollow cylinder, and providing a mesh hinge. The method can also include configuring the proximal fixation hollow cylinder to be implanted transversely into a proximal phalange. The proximal fixation hollow cylinder can be configured with a proximal opening on a side and proximal flanges framing the proximal opening. The distal fixation hollow cylinder can be configured to be implanted transversely into a distal phalange. The distal fixation hollow cylinder can also be configured with a distal opening on a side and distal flanges framing the distal opening. The mesh hinge can be configured to be held by the proximal flanges and the distal flanges to connect the proximal fixation hollow cylinder to the distal fixation hollow cylinder.

In these methods, the configuring can include selecting a size and shape of the components such as the mesh hinge, the rod sleeves, the fixation rods, the bores drilled in the phalanges, the proximal and distal fixation hollow cylinders, and so on. Because the size and shape of bones varies from patient to patient, different sized components may be useful for different patients. In some examples, the length, width, or thickness of the mesh hinge can be selected to be appropriate for a particular patient's anatomy. The various components can also be sized to function correctly together. For example, the mesh hinge can have a thickness selected to fit between the flanges of a proximal fixation hollow cylinder. In another example, the diameter of fixation rods can be selected to fit in the rod sleeves of a mesh hinge. Configuring can also include assembling some of all of the components of the stemless implantable devices for arthroplasty. Some components can be pre-assembled and then implanted in their assembled state. In other examples, components can be temporarily assembled for test fitting, and then disassembled before implanting the device. Any of these processes can be part of configuring the device.

### EXAMPLES

### Example 1—Surgical Methods

According to some embodiments, and as described herein, surgical access and preparation of the implantation site may be achieved through a single mid-lateral incision without disturbing the nearby tendon systems. The transverse implantation method as disclosed herein allows for using minimally invasive surgical access via a mid-lateral incision. The mid-lateral approach avoids the need to interfere with flexor and extensor tendon systems during surgery and allows the joint to be exercised immediately upon removal of sutures without stressing the surgical wound. An exemplary surgical method is as follows:
1) Under tourniquet control, a mid-lateral incision is made over either the radial or ulnar aspect of the proximal interphalangeal joint. A #15 knife is used to incise the skin. Blunt dissection is carried down to the transverse retinacular ligament.
2) The transverse retinacular ligament is sharply divided with a #15 knife. The collateral ligament is sharply divided to expose the joint and allowing for the joint to be “booked open” hinging on the intact collateral ligament.
3) The joint is then “booked open,” hinging on the intact collateral ligament, so that the joint can be examined. If the joint is arthritic or is of poor quality for some other reason, the procedure may continue. In the unlikely event that the joint is found to be of good quality, the soft tissues should be repaired, and the procedure aborted.
4) Large osteophytes are taken down with a Rongeur to smooth the contours of the proximal and middle phalanges. The base of the middle phalange usually requires no further preparation. A sagittal saw (9 mm×15 mm) is then used to remove the articular bearing condyles from the head of the proximal phalange. This cut should be made at the collateral ligament origin/metaphyseal flare of the proximal phalange. The cut should be relatively perpendicular to the bone in the coronal and sagittal planes, but true orthogonal cuts are not required.
5) After removal of the condyles, a guidewire (GW) is placed in the head of the proximal phalange along the axis of rotation. In certain embodiments, a pre-positioning alignment guide to help ensure the proper bone cut and offset for guide pin placement. Such a guide can depend on universal commonalities of the proximal phalange The guidewire should be advanced through the far cortex just far enough to pierce and exit the skin on the other side. A hand-held pin guide/soft tissue protector is then placed over the proximal phalange guidewire (GWp) and used to determine the precise location for placement of a second guidewire which will be located at the base of the middle phalange (GWm). With the aid of multi-planar intraoperative fluoroscopy (mini C-arm), the GWm is placed in the middle phalange and advanced through the far cortex just far enough to pierce and exit the skin on the other side. The hand-held guide is removed, and the adequacy of the guidewire positioning is confirmed using multi-planar fluoroscopy. In certain embodiments, an implant positioning jig is used is placed after the guide pins are passed through the far cortices of the proximal and middle phalanges. This device has motion properties similar to the flexible connector. It attaches to the pins and allow the surgeon to determine the path of motion prior to drilling over the guide pins. Once the pathway is confirmed to be accurate (and free from rotational/angulation abnormalities), the surgeon would overdrill the guide pins with a cannulated drill.
6) Once appropriate guidewire positioning has been confirmed, a trial implant device is affixed to the ends of the guidewires that are exiting the skin. With the trial implant device in place, the position of the finger is clinically assessed at rest, with a wrist tenodesis maneuver (flexing and extending the wrist). The finger is then clinically assessed while the finger is moved through an arc of passive flexion and extension. If there is angulation, rotation, scissoring, or digital malalignment, the guidewires should be repositioned until these issues have been resolved.
7) When the posture and function of the finger is well aligned and balanced, a cannulated drill (2.5 mm) is placed over the GWp and used to drill the receiving hole in the proximal phalange. The drill is advanced through the near cortex and transversely across the proximal phalange head stopping at the far cortex. The far cortex is not perforated with the drill. The drill is then placed on the GWm and used to create the receiving hole in the middle phalange. The drill is advanced through the near cortex and advanced to, but not through the far cortex of the middle phalange. The drill is removed leaving the guidewires in place.
8) The transverse anchor device (TAD) to be inserted into the proximal phalange (TADp) as disclosed herein is then placed over the GWp and inserted into the receiving hole using a cannulated screwdriver. The TADp is advanced until the trailing head is recessed just below the near cortex. The TADp should be turned with the screwdriver to align the guide-mark (bright dot) so that it is facing the middle phalange. The cannulated, channeled transverse anchor is unique. The guidewires and positioning jig are removed after placement of the anchors.
9) The transverse anchor device to be inserted into the distal phalange (TADd) as disclosed herein is then placed over the GWm and inserted into the receiving hole using a cannulated screwdriver. The TADd is advanced until the trailing head is recessed just below the near cortex. The TADd should be turned with a screwdriver to align the guide-mark (bright dot) so that it is facing the guide-mark of the proximal phalange. Aligning the guide-marks so they are facing each-other ensures alignment of the built-in cutting guides. The channel of the anchor also acts as a built-in cutting guide allowing removal of a precise amount of bone with a predetermined “radian” configuration.
10) The trial implant device is removed from the guidewires. The guidewires are then removed. A sagittal saw (or cutting jig) is then used to remove a wedge of bone proximally and distally corresponding to the TADp and TADd built-in cutting guides.
11) A formal implant is selected and opened from a separate, sterile “peel pack”. Preferably, the implants offer different variations of, for example:
   Stiffness (sport)
   Suppleness (art)
   Length (for larger hands)
   Design (woven fiber vs. silicone cushion vs. magnet)
12) The implant rods are press-fitted into the receiving ports of the TADp and TADd. The rods may be further secured by threaded screw caps.
13) Next, the position and passive function of the digit is assessed. In certain embodiments, final adjustments can be made by heating the implant. Note that the amount of heat used during this step must be safe for soft tissues. In some cases, the implant may be heated with a laser. This step permits necessary or desirable corrections to be made in the coronal and sagittal plane.
14) The wound is then irrigated. The transverse retinacular ligament is closed with 5-0 vicryl sutures. The skin is then closed with 4-0 chromic or nylon sutures depending on surgeon preference.
15) The wound may then be dressed with dry gauze and the hand is placed in a volar based plaster splint at the end of the case.
16) Preferably, range of motion exercises under the care of a certified hand therapist begins 7-10 days after surgery if the wound appears to be in a favorable condition for such therapy.
17) In certain embodiments, the system allows for post-insertion positional correction. The material of the flexible connector can be modified for a period of time after insertion to allow for correction of any motion irregularity. This is based on the ability of the material of the flexible connector to be configurable in certain situations.

### Robotic Procedure Option

In lieu of the procedure outlined above, the device may be implanted using robotic equipment designed to replicate the procedural steps and/or outcomes.

### Alignment Check

Additionally, in certain embodiments, a temporary device may be used to check alignment. The temporary device may be set on guidewires protruding through the opposite cortex and skin at the tip of the guidewire, which is preferably 0.1 to 0.6 mm in diameter. Alternatively, the guidewire may be readjusted to sit in a new hole or it could use the same hole as a starting point and inserted so that it has a different trajectory. Using a guidewire with a smaller diameter, or using a jig may permit additional attempts to check the alignment. Pre-alignment may be achieved by using a jig to test the path of motion. In some cases, a temporary provisional implant may be utilized to assess the path of motion before drilling.

The following examples are illustrative of several embodiments of the present technology:
1. A stemless implantable device for arthroplasty, comprising:
   a flexible connector having a proximal rod sleeve integrated in a proximal portion of the flexible connector and a distal rod sleeve integrated in a distal portion of the flexible connector;
   a proximal fixation rod configured to be inserted into the proximal rod sleeve; and
   a distal fixation rod configured to be inserted into the distal rod sleeve.
2. The device of example 1, wherein the proximal fixation rod and the proximal rod sleeve are configured to be implanted in a proximal bore drilled in a proximal phalange, wherein the distal fixation rod and the distal rod sleeve are configured to be implanted in a distal bore drilled in a distal phalange, and wherein the flexible connector is configured to connect the proximal fixation rod and the distal fixation rod within a channel excised through bone along a continuous path between the proximal bore and the distal bore.
3. The device of any preceding example, wherein at least one of the proximal rod sleeve or the distal rod sleeve is open at both ends.
4. The device of any preceding example, wherein at least one of the proximal rod sleeve or the distal rod sleeve is closed at one end, forming a pocket.
5. The device of any preceding example, wherein at least one of the proximal rod sleeve or the distal rod sleeve comprises barbs on an exterior surface operable to securely engage with bone.
6. The device of any preceding example, wherein the flexible connector is stretchable.
7. The device of any preceding example, wherein the flexible connector comprises a material selected from the group consisting of: medical grade plastic, bioactive materials, carbon nanofibers, carbon plates, carbon strips, composite materials, elastomeric polymers, hydroxyapatite (HA) coatings, super-elastic metal alloys, nitinol, internal shape memory alloy, polyetheretherketone (PEEK), silicone, titanium alloys, titanium, polyethylene (PE), engineered polymers, ultra-high molecular weight polyethylene, high molecular weight polyethylene, aromatic polyamide, polymers made to be radiopaque, materials derived therefrom, and combinations thereof.
8. The device of any preceding example, wherein the flexible connector comprises longitudinal strips spaced across a width of the flexible connector and woven together with a flexible material.
9. The device of any preceding example, wherein the longitudinal strips comprise titanium and the flexible material comprises carbon nanofibers.
10. The device of any preceding example, wherein the flexible connector is constructed using a plain weave comprising warp components that have a cross-sectional profile with a greater width than thickness, wherein the warp components run perpendicular to the fixation rods, to achieve a spring mesh which confers lateral stability and alignment in the coronal plane without reliance on joint ligaments.
11. The device of any preceding example, wherein the flexible connector comprises a spring mesh that passively returns to a straight position upon release by a patient's flexor tendon, wherein the mesh hinge prevents contact between bones of the patient's joint.
12. The device of any preceding example, wherein at least one of the proximal fixation rod or the distal fixation rod comprises barbs operable to securely engage the flexible connector.
13. The device of any preceding example, wherein at least one of the proximal fixation rod or the distal fixation rod comprise a smooth exterior surface.
14. The device of any preceding example, wherein at least one of the proximal fixation or the distal fixation rod comprises a biocompatible material.
15. The device of any preceding example, wherein the biocompatible material comprises at least one of a titanium material, a ceramic material, a nickel-based superalloy, a cobalt-chromium alloy, or a nitinol alloy.
16. The device of any preceding example, wherein at least one of the proximal fixation rod or the distal fixation rod comprises an integrated end cap.
17. The device of any preceding example, wherein at least one of a combination of the proximal fixation rod and the proximal rod sleeve or a combination of the distal fixation rod and the distal rod sleeve comprises a tapered end configuration operable to be press fitted into a bore drilled in a phalange.
18. A stemless implantable device for arthroplasty, comprising:
   a proximal fixation hollow cylinder configured to be implanted transversely into a proximal phalange, the proximal fixation hollow cylinder comprising a proximal opening on a side of the proximal fixation hollow cylinder and proximal flanges framing the proximal opening;
   a distal fixation hollow cylinder configured to be implanted transversely into a distal phalange, the distal fixation hollow cylinder comprising a distal opening on a side of the distal fixation hollow cylinder and distal flanges framing the distal opening; and
   a mesh hinge configured to be held by the proximal flanges and the distal flanges to connect the proximal fixation hollow cylinder to the distal fixation hollow cylinder.
19. The device of any preceding example, wherein at least one of the proximal flanges or the distal flanges comprise barbs on an interior surface operable to securely engage the mesh hinge.
20. The device of any preceding example, wherein at least one of the proximal fixation hollow cylinder or the distal fixation hollow cylinder comprises barbs on an exterior surface operable to securely engage with bone.
21. The device of any preceding example, wherein at least one of the proximal fixation hollow cylinder or the distal fixation hollow cylinder comprises an integrated end cap.
22. The device of any preceding example, wherein at least one of the proximal fixation hollow cylinder or the distal fixation hollow cylinder comprises a tapered end configuration operable to be press fitted into a bore drilled in a phalange.
23. The device of any preceding example, wherein at least one of the mesh hinge or the proximal fixation hollow cylinder or the distal fixation hollow cylinder comprises a material selected from the group consisting of: medical grade plastic, bioactive materials, carbon nanofibers, carbon plates, carbon strips, composite materials, elastomeric polymers, hydroxyapatite (HA) coatings, super-elastic metal alloys, nitinol, internal shape memory alloy, polyetheretherketone (PEEK), silicone, titanium alloys, titanium, polyethylene (PE), engineered polymers, ultra-high molecular weight polyethylene, high molecular weight polyethylene, aromatic polyamide, polymers made to be radiopaque, materials derived therefrom, and combinations thereof.
24. The device of any preceding example, wherein the mesh hinge is stretchable.
25. The device of any preceding example, wherein the mesh hinge comprises longitudinal strips spaced across a width of the mesh hinge and woven together with a flexible material.
26. The device of any preceding example, wherein the longitudinal strips comprise titanium and the flexible material comprises carbon nanofibers.
27. The device of any preceding example, wherein the mesh hinge is constructed using a plain weave comprising warp components that have a cross-sectional profile with a greater width than thickness, wherein the warp components run perpendicular to the fixation hollow cylinders, to achieve a spring mesh which confers lateral stability and alignment in the coronal plane without reliance on joint ligaments.
28. The device of any preceding example, wherein the mesh hinge comprises a spring mesh that passively returns to a straight position upon release by a patient's flexor tendon, wherein the mesh hinge prevents contact between bones of the patient's joint.
29. A surgical procedure for implanting a stemless implantable device for arthroplasty, comprising:
   selecting a patient in need of implantation of a stemless implantable device for arthroplasty; and
   implanting into the patient a stemless implantable device for arthroplasty of claim 1.
30. The surgical procedure of claim 29, wherein implanting the device comprises:
   performing a minimally invasive incision at an implant site of a patient;
   preparing a joint capsule and one or more phalangeal bones at the implant site;
   press-fitting the stemless implantable device for arthroplasty into the implant site through the incision; and
   performing a surgical closure of a wound at the implant site.
31. The surgical procedure of example 30, further comprising inserting the proximal fixation rod into the proximal rod sleeve and inserting the distal fixation rod into the distal rod sleeve before press-fitting the stemless implantable device into the implant site.
32. The surgical procedure of any preceding example, wherein the press-fitting comprises press fitting the mesh hinge into the implant site, and wherein the procedure further comprises inserting the proximal fixation rod into the proximal rod sleeve and inserting the distal fixation rod into the distal rod sleeve after press-fitting the mesh hinge into the implant site.
33. A surgical procedure for implanting a stemless implantable device for arthroplasty, comprising:
   selecting a patient in need of implantation of a stemless implantable device for arthroplasty; and
   implanting into the patient a stemless implantable device for arthroplasty of claim 16.
34. The surgical procedure of any preceding example, wherein implanting the device comprises:
   performing a minimally invasive incision at an implant site of a patient;
   preparing a joint capsule and one or more phalangeal bones at the implant site;
   press-fitting the stemless implantable device for arthroplasty into the implant site through the incision; and
   performing a surgical closure of a wound at the implant site.
35. The surgical procedure of any preceding example, further comprising securing the mesh hinge in the proximal flanges and the distal flanges before press-fitting the stemless implantable device into the implant site.
36. A method of configuring an implantable device for arthroplasty, comprising:
   providing a flexible connector having a proximal rod sleeve integrated in a proximal portion of the flexible connector and a distal rod sleeve integrated in a distal portion of the flexible connector;
   providing a proximal fixation rod;
   providing a distal fixation rod;
   configuring the proximal fixation rod to be inserted into the proximal rod sleeve; and
   configuring the distal fixation rod to be inserted into the distal rod sleeve.
37. The method of example 36, further comprising:
   configuring the proximal fixation rod and the proximal rod sleeve to be implanted in a proximal bore drilled in a proximal phalange;
   configuring the distal fixation rod and the distal rod sleeve to be implanted in a distal bore drilled in a distal phalange; and
   configuring the flexible connector to connect the proximal fixation rod and the distal fixation rod through a channel excised through bone along a continuous path between the proximal bore and the distal bore.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Although the disclosure may not expressly disclose that some embodiments or features or examples described herein may be combined with other embodiments or features or examples described herein, this disclosure should be read to describe any such combinations that would be practicable by one of ordinary skill in the art. Indeed, the above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize.

For example, although steps are presented in a given order, alternative embodiments can perform steps in a different order. The various embodiments described herein can also be combined to provide further embodiments.

Furthermore, the described features, structures, characteristics or examples of the present technology may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Moreover, unless the word “or” is expressly limited to mean only a single item exclusive from other items in reference to a list of two or more items, then the use of “or” in such a list is to be interpreted as including (a) any single item in the list, (b) all of the items in the list, or (c) any combination of the items in the list. In other words, the use of “or” in this disclosure should be understood to mean non-exclusive “or” (i.e., “and/or”) unless otherwise indicated herein.

Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A stemless implantable device for arthroplasty, comprising:
a flexible connector having a proximal rod sleeve integrated in a proximal portion of the flexible connector and a distal rod sleeve integrated in a distal portion of the flexible connector;
a proximal fixation rod configured to be inserted into the proximal rod sleeve; and
a distal fixation rod configured to be inserted into the distal rod sleeve.

2. The device of claim 1, wherein the proximal fixation rod and the proximal rod sleeve are configured to be implanted in a proximal bore drilled in a proximal phalange, wherein the distal fixation rod and the distal rod sleeve are configured to be implanted in a distal bore drilled in a distal phalange, and wherein the flexible connector is configured to connect the proximal fixation rod and the distal fixation rod within a channel excised through bone along a continuous path between the proximal bore and the distal bore.

3. The device of claim 1, wherein at least one of the proximal rod sleeve or the distal rod sleeve is open at both ends.

4. The device of claim 1, wherein at least one of the proximal rod sleeve or the distal rod sleeve is closed at one end, forming a pocket.

5. The device of claim 1, wherein at least one of the proximal rod sleeve or the distal rod sleeve comprises barbs on an exterior surface operable to securely engage with bone.

6. The device of claim 1, wherein the flexible connector is stretchable.

7. The device of claim 1, wherein the flexible connector comprises a material selected from the group consisting of: medical grade plastic, bioactive materials, carbon nanofibers, carbon plates, carbon strips, composite materials, elastomeric polymers, hydroxyapatite (HA) coatings, super-elastic metal alloys, nitinol, internal shape memory alloy, polyetheretherketone (PEEK), silicone, titanium alloys, titanium, polyethylene (PE), engineered polymers, ultra-high molecular weight polyethylene, high molecular weight polyethylene, aromatic polyamide, polymers made to be radiopaque, materials derived therefrom, and combinations thereof.

8. The device of claim 1, wherein the flexible connector comprises longitudinal strips spaced across a width of the flexible connector and woven together with a flexible material.

9. The device of claim 8, wherein the longitudinal strips comprise titanium and the flexible material comprises carbon nanofibers.

10. The device of claim 1, wherein the flexible connector is constructed using a plain weave comprising warp components that have a cross-sectional profile with a greater width than thickness, wherein the warp components run perpendicular to the fixation rods, to achieve a spring mesh which confers lateral stability and alignment in the coronal plane without reliance on joint ligaments.

11. The device of claim 1, wherein the flexible connector comprises a spring mesh that passively returns to a straight position upon release by a patient's flexor tendon, wherein the mesh hinge prevents contact between bones of the patient's joint.

12. The device of claim 1, wherein at least one of the proximal fixation rod or the distal fixation rod comprises barbs operable to securely engage the flexible connector.

13. The device of claim 1, wherein at least one of the proximal fixation rod or the distal fixation rod comprise a smooth exterior surface.

14. The device of claim 1, wherein at least one of the proximal fixation or the distal fixation rod comprises a biocompatible material.

15. The device of claim 14, wherein the biocompatible material comprises at least one of a titanium material, a ceramic material, a nickel-based superalloy, a cobalt-chromium alloy, or a nitinol alloy.

16. The device of claim 1, wherein at least one of the proximal fixation rod or the distal fixation rod comprises an integrated end cap.

17. The device of claim 1, wherein at least one of a combination of the proximal fixation rod and the proximal rod sleeve or a combination of the distal fixation rod and the distal rod sleeve comprises a tapered end configuration operable to be press fitted into a bore drilled in a phalange.

18. A surgical procedure for implanting a stemless implantable device for arthroplasty, comprising:
selecting a patient in need of implantation of a stemless implantable device for arthroplasty; and
implanting into the patient a stemless implantable device for arthroplasty of claim 1.

19. The surgical procedure of claim 18, wherein implanting the device comprises:
performing a minimally invasive incision at an implant site of a patient;
preparing a joint capsule and one or more phalangeal bones at the implant site;
press-fitting the stemless implantable device for arthroplasty into the implant site through the incision; and
performing a surgical closure of a wound at the implant site.

20. The surgical procedure of claim 18, further comprising inserting the proximal fixation rod into the proximal rod sleeve and inserting the distal fixation rod into the distal rod sleeve before press-fitting the stemless implantable device into the implant site.

21. The surgical procedure of claim 20, wherein the press-fitting comprises press fitting the mesh hinge into the implant site, and wherein the procedure further comprises inserting the proximal fixation rod into the proximal rod sleeve and inserting the distal fixation rod into the distal rod sleeve after press-fitting the mesh hinge into the implant site.

22. A method of configuring an implantable device for arthroplasty, comprising:
providing a flexible connector having a proximal rod sleeve integrated in a proximal portion of the flexible connector and a distal rod sleeve integrated in a distal portion of the flexible connector;
providing a proximal fixation rod;
providing a distal fixation rod;
configuring the proximal fixation rod to be inserted into the proximal rod sleeve; and
configuring the distal fixation rod to be inserted into the distal rod sleeve.

23. The method of claim 22, further comprising:
configuring the proximal fixation rod and the proximal rod sleeve to be implanted in a proximal bore drilled in a proximal phalange;
configuring the distal fixation rod and the distal rod sleeve to be implanted in a distal bore drilled in a distal phalange; and
configuring the flexible connector to connect the proximal fixation rod and the distal fixation rod through a channel excised through bone along a continuous path between the proximal bore and the distal bore.
